SEQUENCE LISTING (7 sequences)

SEQ ID NO: 1 - protein, 16 aa, synthetic construct
IWIAQELRRIGDEFNA

SEQ ID NO: 2 - protein, 16 aa, synthetic construct
REIGAQLRRMADDLNA

SEQ ID NO: 3 - protein, 16 aa, synthetic construct
RNIARHLAQVGDSMDR

SEQ ID NO: 4 - protein, 16 aa, synthetic construct
VECATQLRRFGDKLNF

SEQ ID NO: 5 - protein, 16 aa, synthetic construct
QRYGRELRRMSDEFEG

SEQ ID NO: 6 - protein, 16 aa, synthetic construct
GQVGRQLAIIGDDINR

SEQ ID NO: 7 - protein, 16 aa, synthetic construct
KKLSECLKRIGDELDS

(19) United States
(12) Patent Application Publication
(10) Pub. No.: US 2025/0289780 A1
(43) Pub. Date: Sep. 18, 2025

(54) N,N'-DIPHENYL-4,4'-BIPHENYLDICARBOXAMIDE ANALOGS AND THEIR USE IN CANCER TREATMENT

(71) Applicant: Board of Regents, The University of Texas System, Austin, TX (US)

(72) Inventor: Jung-Mo Ahn, Plano, TX (US)

(21) Appl. No.: 18/604,733

(22) Filed: Mar. 14, 2024

(51) Int. Cl. C07C 235/64; A61K 31/192

(52) U.S. Cl. CPC C07C 235/64; A61K 31/192

(57) ABSTRACT

In one aspect, the disclosure relates to a for α-helix mimetics that can replicate the α-helices found at protein protein interaction (PPI) interfaces and can selectively discriminate cognate proteins in PPIs. In one aspect, the scaffolds have Formula I

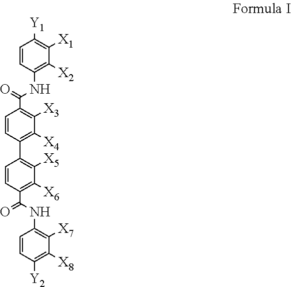

wherein each of X₁-X₈ is individually selected from H, OR₁, NR₁R₂, SR₁, or PR₁R₂;
wherein each of Y₁-Y₂ is individually selected from H, CO₂R₁, C(═O)NR₁R₂, C(═O)R₁, OR₁, NR₁R₂, SR₁, or PR₁R₂; and
wherein each of R₁ and R₂ is individually selected from H, or optionally substituted linear or branched C1-C20 alkyl, C3-C20 aryl, linear or branched C2-C20 alkenyl, linear or branched C2-C20 alkynyl, or C3-C20 arylalkyl.

Also disclosed are methods of making the compounds, pharmaceutical compositions comprising the compounds, and methods of treating cancer using the compounds.

### CROSS REFERENCE TO SEQUENCE LISTING

The genetic components described herein are referred to by sequence identifier numbers (SEQ ID NO). The SEQ ID NOs correspond numerically to the sequence identifiers <400>1, <400>2, etc. The sequence listing in written computer readable format (CRF) as a text file named “292005-1200_SEQUENCE_LISTING.xml” created on Feb. 12, 2024, and having a size of 7,033 bytes, is incorporated by reference in its entirety.

### BACKGROUND

Protein-protein interactions (PPIs) play a key role in numerous biological processes and aberrant PPIs can result in various human diseases. Thus, understanding and modulating PPIs are critical for developing tool compounds and potentially therapeutic candidates. Protein secondary structures, such as α-helix, β-strand, and β-turn structures are responsible for most PPIs. In particular, an α-helix is frequently found at the interface of PPIs and mediates numerous protein functions. Thus, short peptides corresponding to helical protein segments are promising targets to disrupt α-helix-mediated PPIs. However, their utility is compromised by low metabolic stability and poor bioavailability.

An effective approach is the use of α-helix mimetics that are designed to reproduce the structure and function of α-helices. α-Helix mimetics generally comprise of rigid non-peptidic scaffolds to place their substituents in a spatial arrangement mirroring the side chains of an α-helix. Thus, α-helix mimetics have recently attracted considerable attention as potential therapeutic candidates. However, the α-helix mimetics reported to date still have several drawbacks. They were designed based on an ideal α-helix while naturally occurring α-helices in proteins may deviate from the ideal one in the backbone conformation. With the conformational rigidity inherently installed, a scaffold can adopt only a limited range of conformations, which compromises it to accommodate the bound conformations of proteins that are frequently induced or selected by the binding partner at PPI interfaces. The situation can be further complicated when selectively targeting closely related proteins. Such proteins share many structural features but exert distinct biological activities due to subtle differences in their bound conformations.

What is needed is a scaffold that covers as much conformational space as possible in order to effectively replicate the α-helices found at PPI interfaces and to selectively discriminate cognate proteins in PPIs. An ideal scaffold would be capable of making subtle adjustment in presenting its substituents for precise reproduction of naturally occurring helical surfaces. Such a scaffold could be employed to mimic the hydrophobic helical surface of natural proteins such as, for example, the BH3 domains in various pro-apoptotic Bcl-2 family proteins for selectively inhibiting anti-apoptotic members. This scaffold would further be bioavailable and metabolically stable as well as facile to produce. These needs and other needs are satisfied by the present disclosure.

### SUMMARY

In accordance with the purpose(s) of the present disclosure, as embodied and broadly described herein, the disclosure, in one aspect, relates to a for α-helix mimetics that can replicate the α-helices found at protein protein interaction (PPI) interfaces and can selectively discriminate cognate proteins in PPIs. In one aspect, the scaffolds have Formula I

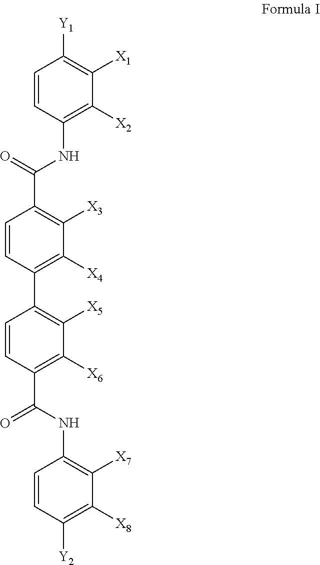

wherein each of X₁-X₈ is individually selected from H, OR₁, NR₁R₂, SR₁, or PR₁R₂;
wherein each of Y₁-Y₂ is individually selected from H, CO₂R₁, C(═O)NR₁R₂, C(═O)R₁, OR₁, NR₁R₂, SR₁, or PR₁R₂;
wherein each of R₁ and R₂ is individually selected from H, linear or branched C1-C20 alkyl, C3-C20 aryl, linear or branched C2-C20 alkenyl, linear or branched C2-C20 alkynyl, or C3-C20 arylalkyl; and
wherein when R₁, R₂, or both R₁ and R₂ are linear or branched C1-C20 alkyl, C3-C20 aryl, linear or branched C2-C20 alkenyl, linear or branched C2-C20 alkynyl, or C3-C20 arylalkyl, R₁, R₂, or both R₁ and R₂ can be optionally substituted with an ether, amine, alkoxy, ester, amide, thiol, phosphine, or any combination thereof.

Also disclosed are methods of making the compounds, pharmaceutical compositions comprising the compounds, and methods of treating cancer using the compounds.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1A shows Structure of an α-helix mimetic based on a biphenyl scaffold I. * corresponds to β-carbon of amino acids. FIG. 1B shows a representative conformation of scaffold I. FIG. 1C shows superimposition of 1 (orange) on an α-helix (green). FIGS. 1Di-1Dii show structures of scaffolds II-VIII and their representative conformations. FIG. 1E shows conformational diversity accessible by a collection of scaffolds I-VIII.

FIG. 2A shows X-ray crystal structure of Bim-BH3 peptide (blue)/Mcl-1 (white) complex.

FIG. 2B shows amino acid sequences of BH3 peptides (SEQ ID NOs. 1-7). Key hydrophobic residues are highlighted in grey. FIG. 2C shows superimposition of BH3 peptides of pro-apoptotic proteins in complex with anti-apoptotic proteins. FIG. 2D shows superimposition of Bim-BH3 peptides in complex with anti-apoptotic proteins.

FIGS. 3A-3B show binding affinity to Bcl-xL, Bcl-2 and Mcl-1 determined by FP assay. FIG. 3A: compounds 1-9 mimicking BH3 peptides of proapoptotic proteins (scaffold I). FIG. 3B: Bim-mimicking compounds 10-16 derived from scaffolds II-VIII.

FIG. 4A shows libraries 1 and 2 of α-helix mimetics based on Bim-mimetic 1. FIG. 4B shows binding affinity of compounds 17-23 to Bcl-xL, Bcl-2 and Mcl-1 determined by FP assay.

FIGS. 5A-5F show cellular activity of mimetics 1, 14, and 23. FIG. 5A: effect of mimetics on the interaction between Bak and antiapoptotic proteins. PC3 cells were incubated in the presence or absence of mimetics (10 μM) for 24 h and cell lysates were analyzed by immunoprecipitation followed by western blotting. FIG. 5B: mitochondrial membrane depolarization by mimetics. PC3 cells that were treated with mimetics, ABT737 or MIM1 were stained with JC-1 dye and analyzed for change in red to green fluorescence intensity ratio of JC-1 dye. FIG. 5C: activation of caspase-3/-9 by mimetics. PC3 cells were treated with different concentrations of mimetics, ABT737, and MIM1 for 24 h and activities of caspase-3/-9 were measured using caspase-specific colorimetric assay kits. FIG. 5D: downregulation of Mcl-1 by siRNA. PC3 cells were transfected with Mcl-1 siRNA (30 μmol) or control siRNA (30 μmol) for 24 h and cell lysates were analyzed by western blotting. FIG. 5E: effect of Mcl-1 knockdown on mimetics-induced caspase activation. Same as for C except for the use of Mcl-1 knockdown PC3 cells. FIG. 5F: effect of Mcl-1 knockdown on mimetics-induced cell growth. PC3 cells were treated with mimetics (10 μM) or MIM1 (10 μM) for 24 h and cell viability was determined using MTT assay.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

Disclosed herein is a scaffold for α-helix mimetics that covers as much conformational space as possible in order to effectively replicate the α-helices found at PPI interfaces and to selectively discriminate cognate proteins in PPIs. Previously, scaffolds such as pyrrolidines, oligoamides, and cyclopropanes were designed to present multiple conformations and applied to mimic α-helix, β-strand and turn structures. The scaffold disclosed herein is capable of making subtle adjustments in presenting its substituents for precise reproduction of naturally occurring helical surfaces. In one aspect, this novel scaffold was employed to mimic the hydrophobic helical surface of the BH3 domains in various pro-apoptotic Bcl-2 family proteins for selectively inhibiting anti-apoptotic members.

In one aspect, disclosed herein is a compound having Formula I or a pharmaceutically acceptable salt thereof

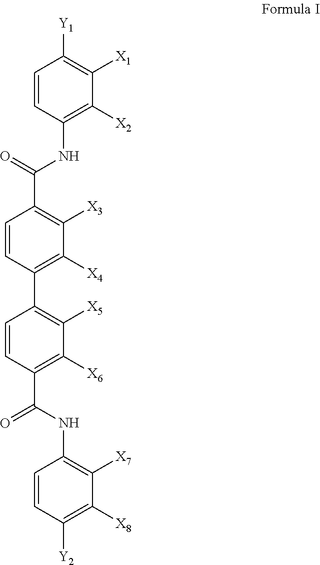

wherein each of X₁-X₈ is individually selected from H, OR₁, NR₁R₂, SR₁, or PR₁R₂;
wherein each of Y₁-Y₂ is individually selected from H, CO₂R₁, C(═O)NR₁R₂, C(═O)R₁, OR₁, NR₁R₂, SR₁, or PR₁R₂;
wherein each of R₁ and R₂ is individually selected from H, linear or branched C1-C20 alkyl, C3-C20 aryl, linear or branched C2-C20 alkenyl, linear or branched C2-C20 alkynyl, or C3-C20 arylalkyl; and
wherein when R₁, R₂, or both R₁ and R₂ are linear or branched C1-C20 alkyl, C3-C20 aryl, linear or branched C2-C20 alkenyl, linear or branched C2-C20 alkynyl, or C3-C20 arylalkyl, R₁, R₂, or both R₁ and R₂ can be optionally substituted with an ether, amine, alkoxy, ester, amide, thiol, phosphine, or any combination thereof.

In some aspects, at least four of X₁-X₈ are H. In an aspect, X₁. X₄, X₅, and Xₐ are H; or X₁. X₄, X₆, and Xₐ are H; or X₂. X₄, X₆, and Xₐ are H; or X₁. X₃, X₅, and Xₐ are H; or X₁. X₃, X₅, and X₇ are H; or X₂. X₄, X₅, and Xₐ are H; or X₂. X₄, X₅, and X₇ are H; or X₁. X₄, X₅, and X₇ are H.

In another aspect, at least four of X₁-X₈ are OR₁. In one aspect, X₂. X₃, X₆, and X₇ are OR₁; or X₂. X₃, X₅, and X₇ are OR₁; or X₁. X₃, X₅, and X₇ are OR₁; or X₂. X₄, X₆, and X₇ are OR₁; or X₂. X₄, X₆, and Xₑ are OR₁; or X₁. X₃, X₆, and X₇ are OR₁; or X₁. X₃, X₆, and Xₐ are OR₁; or X₂. X₃, X₆, and X₈ are OR₁.

In another aspect, Y₁ and Y₂ are CO₂H; or Y₁ is H and Y₂ is CO₂H; or Y₁ is CO₂H and Y₂ is H.

In one aspect, each R₁ is individually selected from sec-butyl, isobutyl, isopropyl, ethyl, isopentyl, methylbutyl, n-pentyl, phenylethyl, benzyl, naphthyl, or n-butyl.

In still another aspect, the compound is selected from

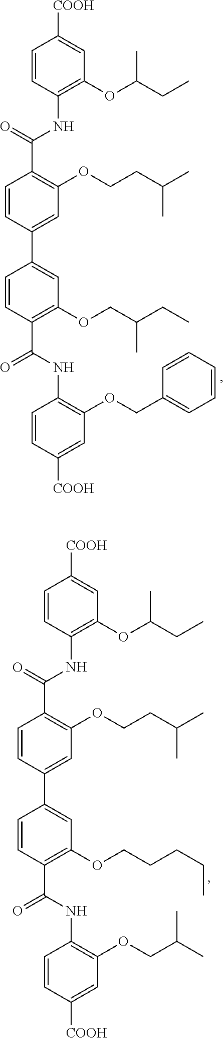 [SMILES: CCC(C)COc1cc(-c2ccc(C(=O)Nc3ccc(C(=O)O)cc3OC(C)CC)c(OCCC(C)C)c2)ccc1C(=O)Nc1ccc(C(=O)O)cc1OCc1ccccc1 ; CCCCCOc1cc(-c2ccc(C(=O)Nc3ccc(C(=O)O)cc3OC(C)CC)c(OCCC(C)C)c2)ccc1C(=O)Nc1ccc(C(=O)O)cc1OCC(C)C]

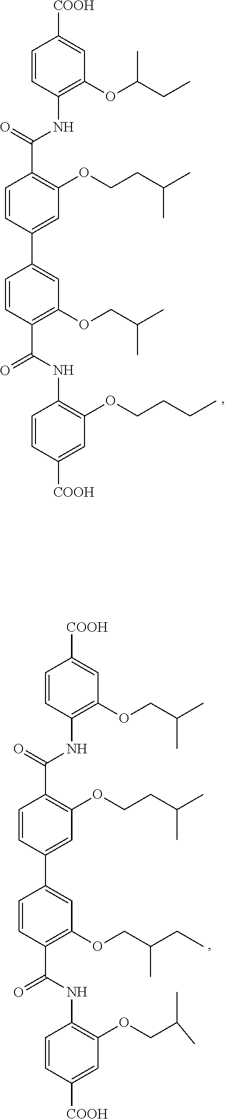 [SMILES: CCCCOc1cc(C(=O)O)ccc1NC(=O)c1ccc(-c2ccc(C(=O)Nc3ccc(C(=O)O)cc3OC(C)CC)c(OCCC(C)C)c2)cc1OCC(C)C ; CCC(C)COc1cc(-c2ccc(C(=O)Nc3ccc(C(=O)O)cc3OCC(C)C)c(OCCC(C)C)c2)ccc1C(=O)Nc1ccc(C(=O)O)cc1OCC(C)C]

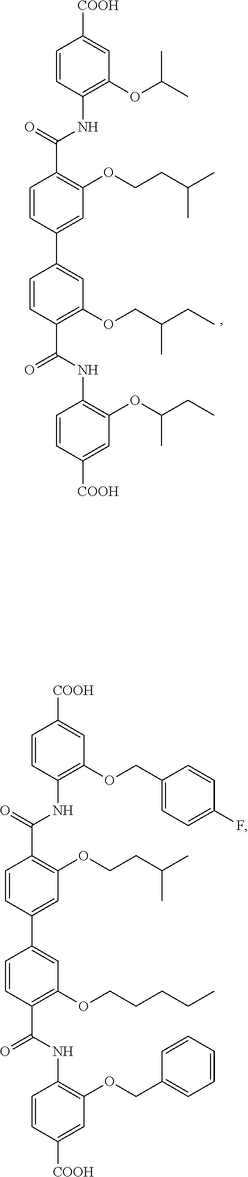 [SMILES: CCC(C)COc1cc(-c2ccc(C(=O)Nc3ccc(C(=O)O)cc3OC(C)C)c(OCCC(C)C)c2)ccc1C(=O)Nc1ccc(C(=O)O)cc1OC(C)CC ; CCCCCOc1cc(-c2ccc(C(=O)Nc3ccc(C(=O)O)cc3OCc3ccc(F)cc3)c(OCCC(C)C)c2)ccc1C(=O)Nc1ccc(C(=O)O)cc1OCc1ccccc1]

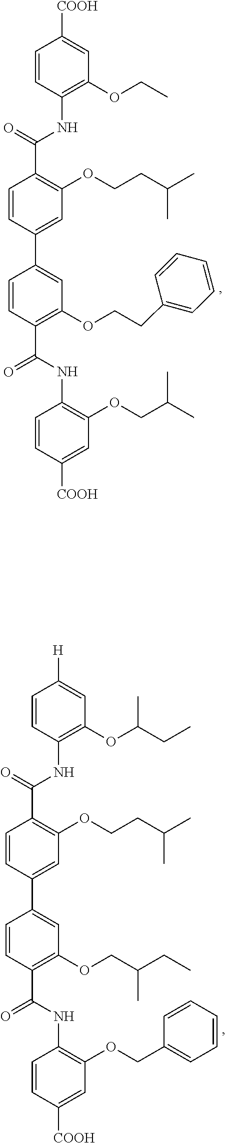 [SMILES: CCOc1cc(C(=O)O)ccc1NC(=O)c1ccc(-c2ccc(C(=O)Nc3ccc(C(=O)O)cc3OCC(C)C)c(OCCc3ccccc3)c2)cc1OCCC(C)C ; CCC(C)COc1cc(-c2ccc(C(=O)Nc3ccccc3OC(C)CC)c(OCCC(C)C)c2)ccc1C(=O)Nc1ccc(C(=O)O)cc1OCc1ccccc1]

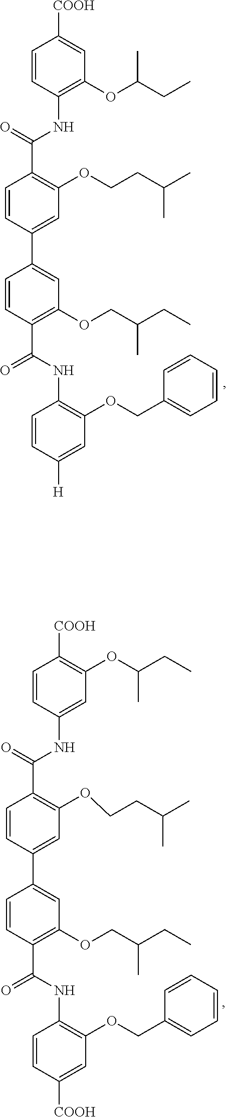 [SMILES: CCC(C)COc1cc(-c2ccc(C(=O)Nc3ccc(C(=O)O)cc3OC(C)CC)c(OCCC(C)C)c2)ccc1C(=O)Nc1ccccc1OCc1ccccc1 ; CCC(C)COc1cc(-c2ccc(C(=O)Nc3ccc(C(=O)O)c(OC(C)CC)c3)c(OCCC(C)C)c2)ccc1C(=O)Nc1ccc(C(=O)O)cc1OCc1ccccc1]

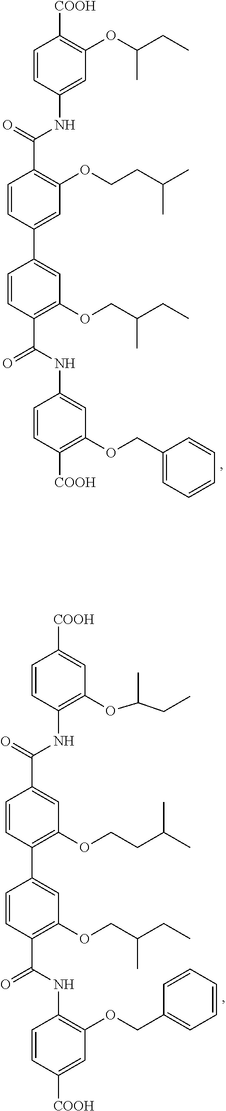 [SMILES: CCC(C)COc1cc(-c2ccc(C(=O)Nc3ccc(C(=O)O)c(OC(C)CC)c3)c(OCCC(C)C)c2)ccc1C(=O)Nc1ccc(C(=O)O)c(OCc2ccccc2)c1 ; CCC(C)COc1cc(-c2ccc(C(=O)Nc3ccc(C(=O)O)cc3OC(C)CC)cc2OCCC(C)C)ccc1C(=O)Nc1ccc(C(=O)O)cc1OCc1ccccc1]

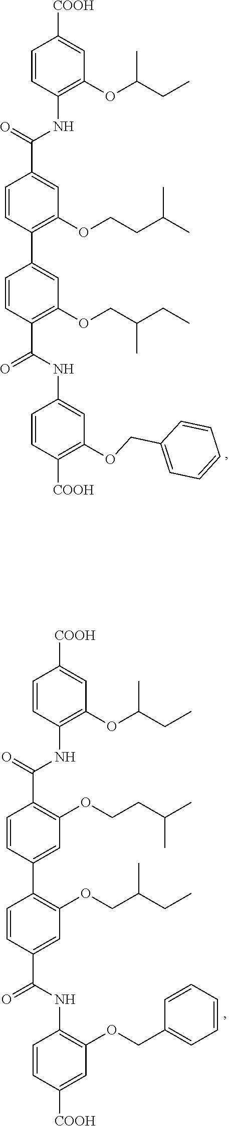 [SMILES: CCC(C)COc1cc(-c2ccc(C(=O)Nc3ccc(C(=O)O)cc3OC(C)CC)cc2OCCC(C)C)ccc1C(=O)Nc1ccc(C(=O)O)c(OCc2ccccc2)c1 ; CCC(C)COc1cc(C(=O)Nc2ccc(C(=O)O)cc2OCc2ccccc2)ccc1-c1ccc(C(=O)Nc2ccc(C(=O)O)cc2OC(C)CC)c(OCCC(C)C)c1]

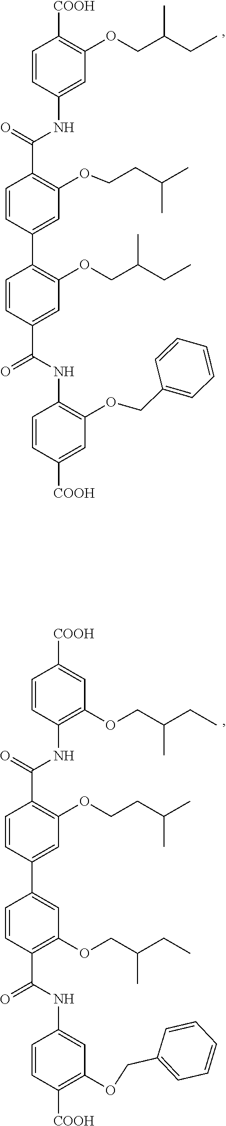 [SMILES: CCC(C)COc1cc(NC(=O)c2ccc(-c3ccc(C(=O)Nc4ccc(C(=O)O)cc4OCc4ccccc4)cc3OCC(C)CC)cc2OCCC(C)C)ccc1C(=O)O ; CCC(C)COc1cc(C(=O)O)ccc1NC(=O)c1ccc(-c2ccc(C(=O)Nc3ccc(C(=O)O)c(OCc4ccccc4)c3)c(OCC(C)CC)c2)cc1OCCC(C)C]

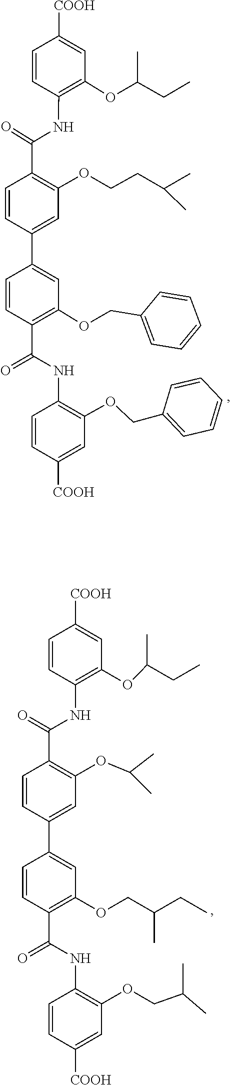 [SMILES: CCC(C)Oc1cc(C(=O)O)ccc1NC(=O)c1ccc(-c2ccc(C(=O)Nc3ccc(C(=O)O)cc3OCc3ccccc3)c(OCc3ccccc3)c2)cc1OCCC(C)C ; CCC(C)COc1cc(-c2ccc(C(=O)Nc3ccc(C(=O)O)cc3OC(C)CC)c(OC(C)C)c2)ccc1C(=O)Nc1ccc(C(=O)O)cc1OCC(C)C]

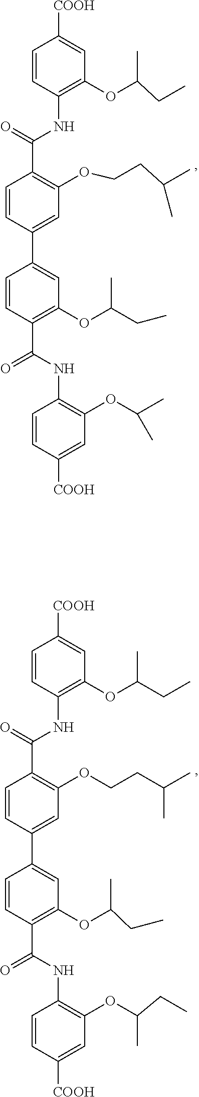 [SMILES: CCC(C)Oc1cc(C(=O)O)ccc1NC(=O)c1ccc(-c2ccc(C(=O)Nc3ccc(C(=O)O)cc3OC(C)C)c(OC(C)CC)c2)cc1OCCC(C)C ; CCC(C)Oc1cc(C(=O)O)ccc1NC(=O)c1ccc(-c2ccc(C(=O)Nc3ccc(C(=O)O)cc3OC(C)CC)c(OC(C)CC)c2)cc1OCCC(C)C]

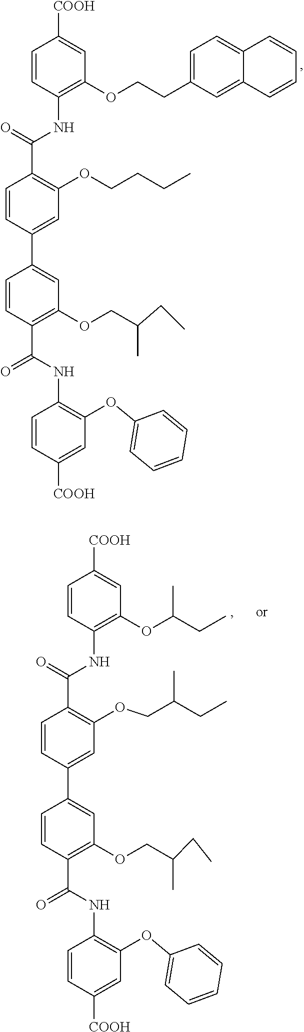 [SMILES: CCCCOc1cc(-c2ccc(C(=O)Nc3ccc(C(=O)O)cc3Oc3ccccc3)c(OCC(C)CC)c2)ccc1C(=O)Nc1ccc(C(=O)O)cc1OCCc1ccc2ccccc2c1 ; CCC(C)COc1cc(-c2ccc(C(=O)Nc3ccc(C(=O)O)cc3OC(C)CC)c(OCC(C)CC)c2)ccc1C(=O)Nc1ccc(C(=O)O)cc1Oc1ccccc1]

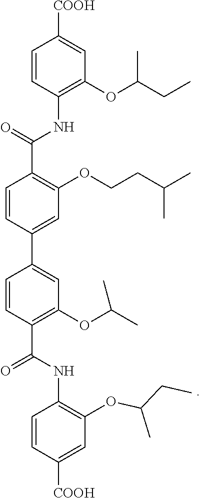 [SMILES: CCC(C)Oc1cc(C(=O)O)ccc1NC(=O)c1ccc(-c2ccc(C(=O)Nc3ccc(C(=O)O)cc3OC(C)CC)c(OC(C)C)c2)cc1OCCC(C)C]

In any of these aspects, the compound binds to at least one anti-apoptotic protein, such as, for example, Bcl-xL, Bcl-2, Mcl-1, or any combination thereof. In one aspect, the compound has an IC₅₀ for Bcl-xL of less than about 10 μM, or of less than about 1 μM, or of less than about 10, 9.5, 9, 8.5, 8, 7.5, 7, 6.5, 6, 6.5, 5.5, 5, 4.5, 4, 3.5, 3, 2.5, 2, 1.5, 1, 0.75, 0.5, 0.25, 0.1 μM, or a combination of any of the foregoing values, or a range encompassing any of the foregoing values.

In another aspect, the compound has an IC₅₀ for Bcl-2 of less than about 10 μM, or less than about 1 μM, or of less than about 0.1 μM, or of less than about 1, 0.95, 0.9, 0.85, 0.8, 0.75, 0.7, 0.65, 0.6, 0.55, 0.5, 0.45, 0.4, 0.35, 0.3, 0.25, 0.2, 0.15, 0.1, 0.075, 0.05, 0.025, 0.01 μM, or a combination of any of the foregoing values, or a range encompassing any of the foregoing values.

In still another aspect, the compound has an IC₅₀ for Mcl-1 of less than about 10 μM, or less than about 2 μM, or of less than about 0.2 μM, or of less than about 2, 1.75, 1.5, 1.25, 1, 0.75, 0.5, 0.25, 0.2, 0.15, 0.1, 0.075, 0.05, 0.025, 0.01 μM, or a combination of any of the foregoing values, or a range encompassing any of the foregoing values.

In some aspects, the compound inhibits two or more anti-apoptotic proteins. In other aspects, the compound is selective for a single anti-apoptotic protein.

In another aspect, disclosed herein is pharmaceutical composition including the compound of Formula I or pharmaceutically acceptable salt thereof. In still another aspect, the pharmaceutical composition includes at least one pharmaceutically acceptable excipient such as, for example, an α-cyclodextrin, a β-cyclodextrin, a γ-cyclodextrin, liposomes, a surfactant, glycerol, a polyethylene glycol, a preservative, water, ethanol, a vegetable oil, saline solution, glucose solution, a phosphate buffer, mannitol, normal serum albumin, sodium chloride solution, Ringer's dextrose, lactated Ringer's, a fixed oil, a chelating agent, or any combination thereof.

Also disclosed herein is method for treating cancer in a subject, the method including at least the step of administering a therapeutically effective amount of the compound, pharmaceutically acceptable salt, or pharmaceutical composition to the subject.

In one aspect, the cancer can be prostate cancer, follicular lymphoma, chronic lymphocytic leukemia, acute lymphocytic leukemia, diffuse large B cell lymphoma, multiple myeloma, acute myeloid leukemia, breast cancer, small cell lung cancer, non-small cell lung cancer, ovarian cancer, neuroblastoma, bladder cancer, colorectal cancer, head and neck cancer, or any combination thereof.

In some aspects, the cancer is a tumor in the subject and the tumor after treatment has a volume of from at least 5% to at least 50% less than the volume of the tumor before treatment, or of 5, 10, 15, 20, 25, 30, 35, 40, 45, or 50% less than the volume of the tumor before treatment, or a combination of any of the foregoing values, or a range encompassing any of the foregoing values. In another aspect, the tumor has a volume of at least 50% or less compared to an equivalent untreated tumor, or of about 50, 55, 60, 65, 70, 75, 80, 85, 90, 95, or 99% less compared to an equivalent untreated tumor, or a combination of any of the foregoing values, or a range encompassing any of the foregoing values. In another aspect, after treatment, the cancer has gone into remission and the tumor is no longer present.

In one aspect, the subject is a mammal such as, for example, a human, horse, cattle, sheep, goat, swine, dog, cat, guinea pig, rat, mouse, or rabbit.

In another aspect, the compound or pharmaceutical composition is administered orally or intravenously. In some aspects, the disclosed method also includes administering to the subject at least one additional anticancer agent.

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a solvent,” “a base,” or “an alkyl group,” include, but are not limited to, mixtures or combinations of two or more such solvents, bases, or alkyl groups, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less' and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the term “effective amount” refers to an amount that is sufficient to achieve the desired modification of a physical property of the composition or material. For example, an “effective amount” of a disclosed compound refers to an amount that is sufficient to achieve the desired improvement in the property modulated by the formulation component, e.g. achieving the desired level of killing of cancer cells. The specific level in terms of wt % in a composition required as an effective amount will depend upon a variety of factors including type of cancer; size of tumor in the subject; method of administration of the compound; age, sex, and body weight of the subject; and other treatments concurrently or previously administered.

As used herein, the terms “optional” or “optionally” means that the subsequently described event or circumstance can or cannot occur, and that the description includes instances where said event or circumstance occurs and instances where it does not.

Unless otherwise specified, temperatures referred to herein are based on atmospheric pressure (i.e. one atmosphere).

As used herein, “IC₅₀,” is intended to refer to the concentration of a substance (e.g., a compound or a drug) that is required for 50% inhibition of a biological process, or component of a process. For example, IC₅₀ refers to the half maximal (50%) inhibitory concentration (IC) of a substance as determined in a suitable assay.

A residue of a chemical species, as used in the specification and concluding claims, refers to the moiety that is the resulting product of the chemical species in a particular reaction scheme or subsequent formulation or chemical product, regardless of whether the moiety is actually obtained from the chemical species. Thus, an ethylene glycol residue in a polyester refers to one or more —OCH₂CH₂O— units in the polyester, regardless of whether ethylene glycol was used to prepare the polyester. Similarly, a sebacic acid residue in a polyester refers to one or more —CO(CH₂)₈CO— moieties in the polyester, regardless of whether the residue is obtained by reacting sebacic acid or an ester thereof to obtain the polyester.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain aspects, unless expressly indicated to the contrary, individual substituents can be further optionally substituted (i.e., further substituted or unsubstituted).

In defining various terms, “A¹,” “A²,” “A³,” and “A⁴” are used herein as generic symbols to represent various specific substituents. These symbols can be any substituent, not limited to those disclosed herein, and when they are defined to be certain substituents in one instance, they can, in another instance, be defined as some other substituents.

The term “aliphatic” or “aliphatic group,” as used herein, denotes a hydrocarbon moiety that may be straight-chain (i.e., unbranched), branched, or cyclic (including fused, bridging, and spirofused polycyclic) and may be completely saturated or may contain one or more units of unsaturation, but which is not aromatic. Unless otherwise specified, aliphatic groups contain 1-20 carbon atoms. Aliphatic groups include, but are not limited to, linear or branched, alkyl, alkenyl, and alkynyl groups, and hybrids thereof such as (cycloalkyl)alkyl, (cycloalkenyl)alkyl or (cycloalkyl)alkenyl.

The term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic or acyclic. The alkyl group can be branched or unbranched. The alkyl group can also be substituted or unsubstituted. For example, the alkyl group can be substituted with one or more groups including, but not limited to, alkyl, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms. The term alkyl group can also be a C1 alkyl, C1-C2 alkyl, C1-C3 alkyl, C1-C4 alkyl, C1-C5 alkyl, C1-C6 alkyl, C1-C7 alkyl, C1-C8 alkyl, C1-C9 alkyl, C1-C10 alkyl, and the like up to and including a C1-C24 alkyl.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” or “haloalkyl” specifically refers to an alkyl group that is substituted with one or more halide, e.g., fluorine, chlorine, bromine, or iodine. Alternatively, the term “monohaloalkyl” specifically refers to an alkyl group that is substituted with a single halide, e.g. fluorine, chlorine, bromine, or iodine. The term “polyhaloalkyl” specifically refers to an alkyl group that is independently substituted with two or more halides, i.e. each halide substituent need not be the same halide as another halide substituent, nor do the multiple instances of a halide substituent need to be on the same carbon. The term “alkoxyalkyl” specifically refers to an alkyl group that is substituted with one or more alkoxy groups, as described below. The term “aminoalkyl” specifically refers to an alkyl group that is substituted with one or more amino groups. The term “hydroxyalkyl” specifically refers to an alkyl group that is substituted with one or more hydroxy groups. When “alkyl” is used in one instance and a specific term such as “hydroxyalkyl” is used in another, it is not meant to imply that the term “alkyl” does not also refer to specific terms such as “hydroxyalkyl” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, norbornyl, and the like. The term “heterocycloalkyl” is a type of cycloalkyl group as defined above, and is included within the meaning of the term “cycloalkyl,” where at least one of the carbon atoms of the ring is replaced with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, cycloalkyl, alkoxy, amino, ether, halide, hydroxy, nitro, silyl, sulfo-oxo, or thiol as described herein.

The term “alkanediyl” as used herein, refers to a divalent saturated aliphatic group, with one or two saturated carbon atom(s) as the point(s) of attachment, a linear or branched, cyclo, cyclic or acyclic structure, no carbon-carbon double or triple bonds, and no atoms other than carbon and hydrogen. The groups, —CH₂— (methylene), —CH₂CH₂—, —CH₂C(CH₃)₂CH₂—, and —CH₂CH₂CH₂— are non-limiting examples of alkanediyl groups.

The terms “alkoxy” and “alkoxyl” as used herein to refer to an alkyl or cycloalkyl group bonded through an ether linkage; that is, an “alkoxy” group can be defined as —OA¹ where A¹ is alkyl or cycloalkyl as defined above. “Alkoxy” also includes polymers of alkoxy groups as just described; that is, an alkoxy can be a polyether such as —OA¹-OA² or —OA¹-(OA²)ₐ-OA³, where “a” is an integer of from 1 to 200 and A¹, A², and A³ are alkyl and/or cycloalkyl groups.

The term “alkenyl” as used herein is a hydrocarbon group of from 2 to 24 carbon atoms with a structural formula containing at least one carbon-carbon double bond. Asymmetric structures such as (A¹A²)C═C(A³A⁴) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkenyl” as used herein is a non-aromatic carbon-based ring composed of at least three carbon atoms and containing at least one carbon-carbon double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, norbornenyl, and the like. The term “heterocycloalkenyl” is a type of cycloalkenyl group as defined above, and is included within the meaning of the term “cycloalkenyl,” where at least one of the carbon atoms of the ring is replaced with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “alkynyl” as used herein is a hydrocarbon group of 2 to 24 carbon atoms with a structural formula containing at least one carbon-carbon triple bond. The alkynyl group can be unsubstituted or substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkynyl” as used herein is a non-aromatic carbon-based ring composed of at least seven carbon atoms and containing at least one carbon-carbon triple bound. Examples of cycloalkynyl groups include, but are not limited to, cycloheptynyl, cyclooctynyl, cyclononynyl, and the like. The term “heterocycloalkynyl” is a type of cycloalkenyl group as defined above, and is included within the meaning of the term “cycloalkynyl,” where at least one of the carbon atoms of the ring is replaced with a heteroatom such as, but not limited to, nitrogen, oxygen, sulfur, or phosphorus. The cycloalkynyl group and heterocycloalkynyl group can be substituted or unsubstituted. The cycloalkynyl group and heterocycloalkynyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “aromatic group” as used herein refers to a ring structure having cyclic clouds of delocalized π electrons above and below the plane of the molecule, where the π clouds contain (4n+2) π electrons. A further discussion of aromaticity is found in Morrison and Boyd, Organic Chemistry, (5th Ed., 1987), Chapter 13, entitled “Aromaticity,” pages 477-497, incorporated herein by reference. The term “aromatic group” is inclusive of both aryl and heteroaryl groups.

The term “aryl” as used herein is a group that contains any carbon-based aromatic group including, but not limited to, benzene, naphthalene, phenyl, biphenyl, anthracene, and the like. The aryl group can be substituted or unsubstituted. The aryl group can be substituted with one or more groups including, but not limited to, alkyl, cycloalkyl, alkoxy, alkenyl, cycloalkenyl, alkynyl, cycloalkynyl, aryl, heteroaryl, aldehyde, —NH₂, carboxylic acid, ester, ether, halide, hydroxy, ketone, azide, nitro, silyl, sulfo-oxo, or thiol as described herein. The term “biaryl” is a specific type of aryl group and is included in the definition of “aryl.” In addition, the aryl group can be a single ring structure or comprise multiple ring structures that are either fused ring structures or attached via one or more bridging groups such as a carbon-carbon bond. For example, biaryl to two aryl groups that are bound together via a fused ring structure, as in naphthalene, or are attached via one or more carbon-carbon bonds, as in biphenyl.

The term “aldehyde” as used herein is represented by the formula —C(O)H. Throughout this specification “C(O)” is a short hand notation for a carbonyl group, i.e., C═O.

The terms “amine” or “amino” as used herein are represented by the formula —NA¹A², where A¹ and A² can be, independently, hydrogen or alkyl, cycloalkyl, alkenyl, cycloalkenyl, alkynyl, cycloalkynyl, aryl, or heteroaryl group as described herein. A specific example of amino is —NH₂.

The term “alkylamino” as used herein is represented by the formula —NH(-alkyl) and —N(-alkyl)₂, where alkyl is a described herein. Representative examples include, but are not limited to, methylamino group, ethylamino group, propylamino group, isopropylamino group, butylamino group, isobutylamino group, (sec-butyl)amino group, (tert-butyl)amino group, pentylamino group, isopentylamino group, (tert-pentyl)amino group, hexylamino group, dimethylamino group, diethylamino group, dipropylamino group, diisopropylamino group, dibutylamino group, diisobutylamino group, di(sec-butyl)amino group, di(tert-butyl)amino group, dipentylamino group, diisopentylamino group, di(tert-pentyl)amino group, dihexylamino group, N-ethyl-N-methylamino group, N-methyl-N-propylamino group, N-ethyl-N-propylamino group and the like.

The term “carboxylic acid” as used herein is represented by the formula —C(O)OH.

The term “ester” as used herein is represented by the formula —OC(O)A¹ or —C(O)OA¹, where A¹ can be alkyl, cycloalkyl, alkenyl, cycloalkenyl, alkynyl, cycloalkynyl, aryl, or heteroaryl group as described herein. The term “polyester” as used herein is represented by the formula -(A¹O(O)C-A²-C(O)O)ₐ— or -(A¹O(O)C-A²-OC(O))ₐ—, where A¹ and A² can be, independently, an alkyl, cycloalkyl, alkenyl, cycloalkenyl, alkynyl, cycloalkynyl, aryl, or heteroaryl group described herein and “a” is an integer from 1 to 500. “Polyester” is as the term used to describe a group that is produced by the reaction between a compound having at least two carboxylic acid groups with a compound having at least two hydroxyl groups.

The term “ether” as used herein is represented by the formula A¹OA², where A¹ and A² can be, independently, an alkyl, cycloalkyl, alkenyl, cycloalkenyl, alkynyl, cycloalkynyl, aryl, or heteroaryl group described herein. The term “polyether” as used herein is represented by the formula -(A¹O-A²O)ₐ—, where A¹ and A² can be, independently, an alkyl, cycloalkyl, alkenyl, cycloalkenyl, alkynyl, cycloalkynyl, aryl, or heteroaryl group described herein and “a” is an integer of from 1 to 500. Examples of polyether groups include polyethylene oxide, polypropylene oxide, and polybutylene oxide.

The terms “halo,” “halogen” or “halide,” as used herein can be used interchangeably and refer to F, Cl, Br, or I.

The terms “pseudohalide,” “pseudohalogen” or “pseudohalo,” as used herein can be used interchangeably and refer to functional groups that behave substantially similar to halides. Such functional groups include, by way of example, cyano, thiocyanato, azido, trifluoromethyl, trifluoromethoxy, perfluoroalkyl, and perfluoroalkoxy groups.

The term “heteroalkyl” as used herein refers to an alkyl group containing at least one heteroatom. Suitable heteroatoms include, but are not limited to, O, N, Si, P and S, wherein the nitrogen, phosphorous and sulfur atoms are optionally oxidized, and the nitrogen heteroatom is optionally quaternized. Heteroalkyls can be substituted as defined above for alkyl groups.

The term “heteroaryl” as used herein refers to an aromatic group that has at least one heteroatom incorporated within the ring of the aromatic group. Examples of heteroatoms include, but are not limited to, nitrogen, oxygen, sulfur, and phosphorus, where N-oxides, sulfur oxides, and dioxides are permissible heteroatom substitutions. The heteroaryl group can be substituted or unsubstituted. The heteroaryl group can be substituted with one or more groups including, but not limited to, alkyl, cycloalkyl, alkoxy, amino, ether, halide, hydroxy, nitro, silyl, sulfo-oxo, or thiol as described herein. Heteroaryl groups can be monocyclic, or alternatively fused ring systems. Heteroaryl groups include, but are not limited to, furyl, imidazolyl, pyrimidinyl, tetrazolyl, thienyl, pyridinyl, pyrrolyl, N-methylpyrrolyl, quinolinyl, isoquinolinyl, pyrazolyl, triazolyl, thiazolyl, oxazolyl, isoxazolyl, oxadiazolyl, thiadiazolyl, isothiazolyl, pyridazinyl, pyrazinyl, benzofuranyl, benzodioxolyl, benzothiophenyl, indolyl, indazolyl, benzimidazolyl, imidazopyridinyl, pyrazolopyridinyl, and pyrazolopyrimidinyl. Further not limiting examples of heteroaryl groups include, but are not limited to, pyridinyl, pyridazinyl, pyrimidinyl, pyrazinyl, thiophenyl, pyrazolyl, imidazolyl, benzo[d]oxazolyl, benzo[d]thiazolyl, quinolinyl, quinazolinyl, indazolyl, imidazo[1,2-b]pyridazinyl, imidazo[1,2-a]pyrazinyl, benzo[c][1,2,5]thiadiazolyl, benzo[c][1,2,5]oxadiazolyl, and pyrido[2,3-b]pyrazinyl.

The terms “heterocycle” or “heterocyclyl,” as used herein can be used interchangeably and refer to single and multi-cyclic aromatic or non-aromatic ring systems in which at least one of the ring members is other than carbon. Thus, the term is inclusive of, but not limited to, “heterocycloalkyl,” “heteroaryl,” “bicyclic heterocycle,” and “polycyclic heterocycle.” Heterocycle includes pyridine, pyrimidine, furan, thiophene, pyrrole, isoxazole, isothiazole, pyrazole, oxazole, thiazole, imidazole, oxazole, including, 1,2,3-oxadiazole, 1,2,5-oxadiazole and 1,3,4-oxadiazole, thiadiazole, including, 1,2,3-thiadiazole, 1,2,5-thiadiazole, and 1,3,4-thiadiazole, triazole, including, 1,2,3-triazole, 1,3,4-triazole, tetrazole, including 1,2,3,4-tetrazole and 1,2,4,5-tetrazole, pyridazine, pyrazine, triazine, including 1,2,4-triazine and 1,3,5-triazine, tetrazine, including 1,2,4,5-tetrazine, pyrrolidine, piperidine, piperazine, morpholine, azetidine, tetrahydropyran, tetrahydrofuran, dioxane, and the like. The term heterocyclyl group can also be a C2 heterocyclyl, C2-C3 heterocyclyl, C2-C4 heterocyclyl, C2-C5 heterocyclyl, C2-C6 heterocyclyl, C2-C7 heterocyclyl, C2-C8 heterocyclyl, C2-C9 heterocyclyl, C2-C10 heterocyclyl, C2-C11 heterocyclyl, and the like up to and including a C2-C18 heterocyclyl. For example, a C2 heterocyclyl comprises a group which has two carbon atoms and at least one heteroatom, including, but not limited to, aziridinyl, diazetidinyl, dihydrodiazetyl, oxiranyl, thiiranyl, and the like. Alternatively, for example, a C5 heterocyclyl comprises a group which has five carbon atoms and at least one heteroatom, including, but not limited to, piperidinyl, tetrahydropyranyl, tetrahydrothiopyranyl, diazepanyl, pyridinyl, and the like. It is understood that a heterocyclyl group may be bound either through a heteroatom in the ring, where chemically possible, or one of carbons comprising the heterocyclyl ring.

The term “bicyclic heterocycle” or “bicyclic heterocyclyl” as used herein refers to a ring system in which at least one of the ring members is other than carbon. Bicyclic heterocyclyl encompasses ring systems wherein an aromatic ring is fused with another aromatic ring, or wherein an aromatic ring is fused with a non-aromatic ring. Bicyclic heterocyclyl encompasses ring systems wherein a benzene ring is fused to a 5- or a 6-membered ring containing 1, 2 or 3 ring heteroatoms or wherein a pyridine ring is fused to a 5- or a 6-membered ring containing 1, 2 or 3 ring heteroatoms. Bicyclic heterocyclic groups include, but are not limited to, indolyl, indazolyl, pyrazolo[1,5-a]pyridinyl, benzofuranyl, quinolinyl, quinoxalinyl, 1,3-benzodioxolyl, 2,3-dihydro-1,4-benzodioxinyl, 3,4-dihydro-2H-chromenyl, 1H-pyrazolo[4,3-c]pyridin-3-yl; 1H-pyrrolo[3,2-b]pyridin-3-yl; and 1H-pyrazolo[3,2-b]pyridin-3-yl.

The term “heterocycloalkyl” as used herein refers to an aliphatic, partially unsaturated or fully saturated, 3- to 14-membered ring system, including single rings of 3 to 8 atoms and bi- and tricyclic ring systems. The heterocycloalkyl ring-systems include one to four heteroatoms independently selected from oxygen, nitrogen, and sulfur, wherein a nitrogen and sulfur heteroatom optionally can be oxidized and a nitrogen heteroatom optionally can be substituted. Representative heterocycloalkyl groups include, but are not limited to, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl.

The term “hydroxyl” or “hydroxy” as used herein is represented by the formula —OH.

The term “ketone” as used herein is represented by the formula A¹C(O)A², where A¹ and A² can be, independently, an alkyl, cycloalkyl, alkenyl, cycloalkenyl, alkynyl, cycloalkynyl, aryl, or heteroaryl group as described herein.

The term “azide” or “azido” as used herein is represented by the formula —N₃.

The term “nitro” as used herein is represented by the formula —NO₂.

The term “nitrile” or “cyano” as used herein is represented by the formula —CN.

The term “silyl” as used herein is represented by the formula —S¹A¹A²A³, where A¹, A², and A³ can be, independently, hydrogen or an alkyl, cycloalkyl, alkoxy, alkenyl, cycloalkenyl, alkynyl, cycloalkynyl, aryl, or heteroaryl group as described herein.

The term “sulfo-oxo” as used herein is represented by the formulas —S(O)A¹, —S(O)₂A¹, —OS(O)₂A¹, or —OS(O)₂OA¹, where A¹ can be hydrogen or an alkyl, cycloalkyl, alkenyl, cycloalkenyl, alkynyl, cycloalkynyl, aryl, or heteroaryl group as described herein. Throughout this specification “S(O)” is a short hand notation for S═O. The term “sulfonyl” is used herein to refer to the sulfo-oxo group represented by the formula —S(O)₂A¹, where A¹ can be hydrogen or an alkyl, cycloalkyl, alkenyl, cycloalkenyl, alkynyl, cycloalkynyl, aryl, or heteroaryl group as described herein. The term “sulfone” as used herein is represented by the formula A'S(O)₂A², where A¹ and A² can be, independently, an alkyl, cycloalkyl, alkenyl, cycloalkenyl, alkynyl, cycloalkynyl, aryl, or heteroaryl group as described herein. The term “sulfoxide” as used herein is represented by the formula A'S(O)A², where A¹ and A² can be, independently, an alkyl, cycloalkyl, alkenyl, cycloalkenyl, alkynyl, cycloalkynyl, aryl, or heteroaryl group as described herein.

The term “thiol” as used herein is represented by the formula —SH.

“R¹,” “R²,” “R³,” . . . “Rⁿ,” where n is an integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. In is also contemplated that, in certain aspects, unless expressly indicated to the contrary, individual substituents can be further optionally substituted (i.e., further substituted or unsubstituted).

The term “stable,” as used herein, refers to compounds that are not substantially altered when subjected to conditions to allow for their production, detection, and, in certain aspects, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR—, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, =NR*, =NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₀₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R^{●} include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

The term “leaving group” refers to an atom (or a group of atoms) with electron withdrawing ability that can be displaced as a stable species, taking with it the bonding electrons. Examples of suitable leaving groups include halides and sulfonate esters, including, but not limited to, triflate, mesylate, tosylate, and brosylate.

The terms “hydrolysable group” and “hydrolysable moiety” refer to a functional group capable of undergoing hydrolysis, e.g., under basic or acidic conditions. Examples of hydrolysable residues include, without limitation, acid halides, activated carboxylic acids, and various protecting groups known in the art (see, for example, “Protective Groups in Organic Synthesis,” T. W. Greene, P. G. M. Wuts, Wiley-Interscience, 1999).

The term “organic residue” defines a carbon containing residue, i.e., a residue comprising at least one carbon atom, and includes but is not limited to the carbon-containing groups, residues, or radicals defined hereinabove. Organic residues can contain various heteroatoms, or be bonded to another molecule through a heteroatom, including oxygen, nitrogen, sulfur, phosphorus, or the like. Examples of organic residues include but are not limited alkyl or substituted alkyls, alkoxy or substituted alkoxy, mono or di-substituted amino, amide groups, etc. Organic residues can preferably comprise 1 to 18 carbon atoms, 1 to 15, carbon atoms, 1 to 12 carbon atoms, 1 to 8 carbon atoms, 1 to 6 carbon atoms, or 1 to 4 carbon atoms. In a further aspect, an organic residue can comprise 2 to 18 carbon atoms, 2 to 15, carbon atoms, 2 to 12 carbon atoms, 2 to 8 carbon atoms, 2 to 4 carbon atoms, or 2 to 4 carbon atoms.

A very close synonym of the term “residue” is the term “radical,” which as used in the specification and concluding claims, refers to a fragment, group, or substructure of a molecule described herein, regardless of how the molecule is prepared. For example, a 2,4-thiazolidinedione radical in a particular compound has the structure:

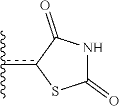

regardless of whether thiazolidinedione is used to prepare the compound. In some embodiments the radical (for example an alkyl) can be further modified (i.e., substituted alkyl) by having bonded thereto one or more “substituent radicals.” The number of atoms in a given radical is not critical to the present invention unless it is indicated to the contrary elsewhere herein.

“Organic radicals,” as the term is defined and used herein, contain one or more carbon atoms. An organic radical can have, for example, 1-26 carbon atoms, 1-18 carbon atoms, 1-12 carbon atoms, 1-8 carbon atoms, 1-6 carbon atoms, or 1-4 carbon atoms. In a further aspect, an organic radical can have 2-26 carbon atoms, 2-18 carbon atoms, 2-12 carbon atoms, 2-8 carbon atoms, 2-6 carbon atoms, or 2-4 carbon atoms. Organic radicals often have hydrogen bound to at least some of the carbon atoms of the organic radical. One example of an organic radical that comprises no inorganic atoms is a 5, 6, 7, 8-tetrahydro-2-naphthyl radical. In some embodiments, an organic radical can contain 1-10 inorganic heteroatoms bound thereto or therein, including halogens, oxygen, sulfur, nitrogen, phosphorus, and the like. Examples of organic radicals include but are not limited to an alkyl, substituted alkyl, cycloalkyl, substituted cycloalkyl, mono-substituted amino, di-substituted amino, acyloxy, cyano, carboxy, carboalkoxy, alkylcarboxamide, substituted alkylcarboxamide, dialkylcarboxamide, substituted dialkylcarboxamide, alkylsulfonyl, alkylsulfinyl, thioalkyl, thiohaloalkyl, alkoxy, substituted alkoxy, haloalkyl, haloalkoxy, aryl, substituted aryl, heteroaryl, heterocyclic, or substituted heterocyclic radicals, wherein the terms are defined elsewhere herein. A few non-limiting examples of organic radicals that include heteroatoms include alkoxy radicals, trifluoromethoxy radicals, acetoxy radicals, dimethylamino radicals and the like.

“Inorganic radicals,” as the term is defined and used herein, contain no carbon atoms and therefore comprise only atoms other than carbon. Inorganic radicals comprise bonded combinations of atoms selected from hydrogen, nitrogen, oxygen, silicon, phosphorus, sulfur, selenium, and halogens such as fluorine, chlorine, bromine, and iodine, which can be present individually or bonded together in their chemically stable combinations. Inorganic radicals have 10 or fewer, or preferably one to six or one to four inorganic atoms as listed above bonded together. Examples of inorganic radicals include, but not limited to, amino, hydroxy, halogens, nitro, thiol, sulfate, phosphate, and like commonly known inorganic radicals. The inorganic radicals do not have bonded therein the metallic elements of the periodic table (such as the alkali metals, alkaline earth metals, transition metals, lanthanide metals, or actinide metals), although such metal ions can sometimes serve as a pharmaceutically acceptable cation for anionic inorganic radicals such as a sulfate, phosphate, or like anionic inorganic radical. Inorganic radicals do not comprise metalloids elements such as boron, aluminum, gallium, germanium, arsenic, tin, lead, or tellurium, or the noble gas elements, unless otherwise specifically indicated elsewhere herein.

Compounds described herein can contain one or more double bonds and, thus, potentially give rise to cis/trans (E/Z) isomers, as well as other conformational isomers. Unless stated to the contrary, the invention includes all such possible isomers, as well as mixtures of such isomers.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture. Compounds described herein can contain one or more asymmetric centers and, thus, potentially give rise to diastereomers and optical isomers. Unless stated to the contrary, the present invention includes all such possible diastereomers as well as their racemic mixtures, their substantially pure resolved enantiomers, all possible geometric isomers, and pharmaceutically acceptable salts thereof. Mixtures of stereoisomers, as well as isolated specific stereoisomers, are also included. During the course of the synthetic procedures used to prepare such compounds, or in using racemization or epimerization procedures known to those skilled in the art, the products of such procedures can be a mixture of stereoisomers.

Many organic compounds exist in optically active forms having the ability to rotate the plane of plane-polarized light. In describing an optically active compound, the prefixes D and L or R and S are used to denote the absolute configuration of the molecule about its chiral center(s). The prefixes d and l or (+) and (−) are employed to designate the sign of rotation of plane-polarized light by the compound, with (−) or meaning that the compound is levorotatory. A compound prefixed with (+) or d is dextrorotatory. For a given chemical structure, these compounds, called stereoisomers, are identical except that they are non-superimposable mirror images of one another. A specific stereoisomer can also be referred to as an enantiomer, and a mixture of such isomers is often called an enantiomeric mixture. A 50:50 mixture of enantiomers is referred to as a racemic mixture. Many of the compounds described herein can have one or more chiral centers and therefore can exist in different enantiomeric forms. If desired, a chiral carbon can be designated with an asterisk (*). When bonds to the chiral carbon are depicted as straight lines in the disclosed formulas, it is understood that both the (R) and (S) configurations of the chiral carbon, and hence both enantiomers and mixtures thereof, are embraced within the formula. As is used in the art, when it is desired to specify the absolute configuration about a chiral carbon, one of the bonds to the chiral carbon can be depicted as a wedge (bonds to atoms above the plane) and the other can be depicted as a series or wedge of short parallel lines is (bonds to atoms below the plane). The Cahn-Ingold-Prelog system can be used to assign the (R) or (S) configuration to a chiral carbon.

Compounds described herein comprise atoms in both their natural isotopic abundance and in non-natural abundance. The disclosed compounds can be isotopically-labeled or isotopically-substituted compounds identical to those described, but for the fact that one or more atoms are replaced by an atom having an atomic mass or mass number different from the atomic mass or mass number typically found in nature. Examples of isotopes that can be incorporated into compounds of the invention include isotopes of hydrogen, carbon, nitrogen, oxygen, sulfur, fluorine and chlorine, such as ²H, ³H, ¹³C, ¹⁴C ¹⁵N, ¹⁸O, ¹⁷O, ³⁵S, ¹⁸F, and ³⁶Cl, respectively. Compounds further comprise prodrugs thereof and pharmaceutically acceptable salts of said compounds or of said prodrugs which contain the aforementioned isotopes and/or other isotopes of other atoms are within the scope of this invention. Certain isotopically-labeled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of the present invention and prodrugs thereof can generally be prepared by carrying out the procedures below, by substituting a readily available isotopically labeled reagent for a non-isotopically labeled reagent.

The compounds described in the invention can be present as a solvate. In some cases, the solvent used to prepare the solvate is an aqueous solution, and the solvate is then often referred to as a hydrate. The compounds can be present as a hydrate, which can be obtained, for example, by crystallization from a solvent or from aqueous solution. In this connection, one, two, three or any arbitrary number of solvent or water molecules can combine with the compounds according to the invention to form solvates and hydrates. Unless stated to the contrary, the invention includes all such possible solvates.

The term “co-crystal” means a physical association of two or more molecules which owe their stability through non-covalent interaction. One or more components of this molecular complex provide a stable framework in the crystalline lattice. In certain instances, the guest molecules are incorporated in the crystalline lattice as anhydrates or solvates, see e.g. “Crystal Engineering of the Composition of Pharmaceutical Phases. Do Pharmaceutical Co-crystals Represent a New Path to Improved Medicines?” Almarasson, O., et al., The Royal Society of Chemistry, 1889-1896, 2004. Examples of co-crystals include p-toluenesulfonic acid and benzenesulfonic acid.

It is also appreciated that certain compounds described herein can be present as an equilibrium of tautomers. For example, ketones with an α-hydrogen can exist in an equilibrium of the keto form and the enol form.

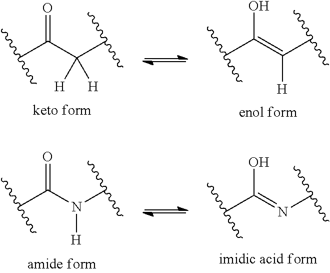 [SMILES: *CC(*)=O ; */C=C(/*)O ; *NC(*)=O ; */N=C(/*)O]

Likewise, amides with an N-hydrogen can exist in an equilibrium of the amide form and the imidic acid form. Unless stated to the contrary, the invention includes all such possible tautomers.

It is known that chemical substances form solids which are present in different states of order which are termed polymorphic forms or modifications. The different modifications of a polymorphic substance can differ greatly in their physical properties. The compounds according to the invention can be present in different polymorphic forms, with it being possible for particular modifications to be metastable. Unless stated to the contrary, the invention includes all such possible polymorphic forms.

In some aspects, a structure of a compound can be represented by a formula:

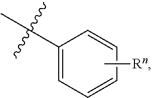

which is understood to be equivalent to a formula:

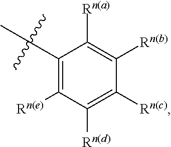

wherein n is typically an integer. That is, Rⁿ is understood to represent five independent substituents, R^{n(a)}, R^{n(b)}, R^{n(c)}, R^{n(d)}, and R^{n(e)}. By “independent substituents,” it is meant that each R substituent can be independently defined. For example, if in one instance R^{n(a)} is halogen, then R^{n(b)} is not necessarily halogen in that instance.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

### Pharmaceutical Compositions

As used herein, “administering” can refer to an administration that is oral, topical, intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravireal, intracerebral, and intracerebroventricular, intratympanic, intracochlear, rectal, vaginal, by inhalation, by catheters, stents or via an implanted reservoir or other device that administers, either actively or passively (e.g. by diffusion) a composition the perivascular space and adventitia. For example a medical device such as a stent can contain a composition or formulation disposed on its surface, which can then dissolve or be otherwise distributed to the surrounding tissue and cells. The term “parenteral” can include subcutaneous, intravenous, intramuscular, intra-articular, intra-synovial, intrasternal, intrathecal, intrahepatic, intralesional, and intracranial injections or infusion techniques. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, “therapeutic agent” can refer to any substance, compound, molecule, and the like, which can be biologically active or otherwise can induce a pharmacologic, immunogenic, biologic and/or physiologic effect on a subject to which it is administered to by local and/or systemic action. A therapeutic agent can be a primary active agent, or in other words, the component(s) of a composition to which the whole or part of the effect of the composition is attributed. A therapeutic agent can be a secondary therapeutic agent, or in other words, the component(s) of a composition to which an additional part and/or other effect of the composition is attributed. The term therefore encompasses those compounds or chemicals traditionally regarded as drugs, vaccines, and biopharmaceuticals including molecules such as proteins, peptides, hormones, nucleic acids, gene constructs and the like. Examples of therapeutic agents are described in well-known literature references such as the Merck Index (14th edition), the Physicians' Desk Reference (64th edition), and The Pharmacological Basis of Therapeutics (12th edition), and they include, without limitation, medicaments; vitamins; mineral supplements; substances used for the treatment, prevention, diagnosis, cure or mitigation of a disease or illness; substances that affect the structure or function of the body, or pro-drugs, which become biologically active or more active after they have been placed in a physiological environment. For example, the term “therapeutic agent” includes compounds or compositions for use in all of the major therapeutic areas including, but not limited to, adjuvants; anti-infectives such as antibiotics and antiviral agents; analgesics and analgesic combinations, anorexics, anti-inflammatory agents, anti-epileptics, local and general anesthetics, hypnotics, sedatives, antipsychotic agents, neuroleptic agents, antidepressants, anxiolytics, antagonists, neuron blocking agents, anticholinergic and cholinomimetic agents, antimuscarinic and muscarinic agents, antiadrenergics, antiarrhythmics, antihypertensive agents, hormones, and nutrients, antiarthritics, antiasthmatic agents, anticonvulsants, antihistamines, antinauseants, antineoplastics, antipruritics, antipyretics; antispasmodics, cardiovascular preparations (including calcium channel blockers, beta-blockers, beta-agonists and antiarrythmics), antihypertensives, diuretics, vasodilators; central nervous system stimulants; cough and cold preparations; decongestants; diagnostics; hormones; bone growth stimulants and bone resorption inhibitors; immunosuppressives; muscle relaxants; psychostimulants; sedatives; tranquilizers; proteins, peptides, and fragments thereof (whether naturally occurring, chemically synthesized or recombinantly produced); and nucleic acid molecules (polymeric forms of two or more nucleotides, either ribonucleotides (RNA) or deoxyribonucleotides (DNA) including both double- and single-stranded molecules, gene constructs, expression vectors, antisense molecules and the like), small molecules (e.g., doxorubicin) and other biologically active macromolecules such as, for example, proteins and enzymes. The agent may be a biologically active agent used in medical, including veterinary, applications and in agriculture, such as with plants, as well as other areas. The term therapeutic agent also includes without limitation, medicaments; vitamins; mineral supplements; substances used for the treatment, prevention, diagnosis, cure or mitigation of disease or illness; or substances which affect the structure or function of the body; or pro-drugs, which become biologically active or more active after they have been placed in a predetermined physiological environment.

As used herein, “attached” can refer to covalent or non-covalent interaction between two or more molecules. Non-covalent interactions can include ionic bonds, electrostatic interactions, van der Walls forces, dipole-dipole interactions, dipole-induced-dipole interactions, London dispersion forces, hydrogen bonding, halogen bonding, electromagnetic interactions, π-π interactions, cation-π interactions, anion-π interactions, polar π-interactions, and hydrophobic effects.

As used interchangeably herein, “subject,” “individual,” or “patient” can refer to a vertebrate organism, such as a mammal (e.g. human). “Subject” can also refer to a cell, a population of cells, a tissue, an organ, or an organism, preferably to human and constituents thereof.

As used herein, the terms “treating” and “treatment” can refer generally to obtaining a desired pharmacological and/or physiological effect. The effect can be, but does not necessarily have to be, prophylactic in terms of preventing or partially preventing a disease, symptom or condition thereof, such as prostate cancer or another cancer. The effect can be therapeutic in terms of a partial or complete cure of a disease, condition, symptom or adverse effect attributed to the disease, disorder, or condition. The term “treatment” as used herein can include any treatment of cancer in a subject, particularly a human and can include any one or more of the following: (a) preventing the disease from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, e.g., such as treating the pain of a subject by administration of an analgesic agent even though such agent does not treat the cause of the pain.

As used herein, “dose,” “unit dose,” or “dosage” can refer to physically discrete units suitable for use in a subject, each unit containing a predetermined quantity of a disclosed compound and/or a pharmaceutical composition thereof calculated to produce the desired response or responses in association with its administration.

As used herein, “therapeutic” can refer to treating, healing, and/or ameliorating a disease, disorder, condition, or side effect, or to decreasing in the rate of advancement of a disease, disorder, condition, or side effect.

As used herein, “effective amount” can refer to the amount of a disclosed compound or pharmaceutical composition provided herein that is sufficient to effect beneficial or desired biological, emotional, medical, or clinical response of a cell, tissue, system, animal, or human. An effective amount can be administered in one or more administrations, applications, or dosages. The term can also include within its scope amounts effective to enhance or restore to substantially normal physiological function.

As used herein, the term “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors within the knowledge and expertise of the health practitioner and which may be well known in the medical arts. In the case of treating a particular disease or condition, in some instances, the desired response can be inhibiting the progression of the disease or condition. This may involve only slowing the progression of the disease temporarily. However, in other instances, it may be desirable to halt the progression of the disease permanently. This can be monitored by routine diagnostic methods known to one of ordinary skill in the art for any particular disease. The desired response to treatment of the disease or condition also can be delaying the onset or even preventing the onset of the disease or condition.

For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. It is generally preferred that a maximum dose of the pharmacological agents of the invention (alone or in combination with other therapeutic agents) be used, that is, the highest safe dose according to sound medical judgment. It will be understood by those of ordinary skill in the art however, that a patient may insist upon a lower dose or tolerable dose for medical reasons, psychological reasons or for virtually any other reasons.

A response to a therapeutically effective dose of a disclosed compound and/or pharmaceutical composition, for example, can be measured by determining the physiological effects of the treatment or medication, such as the decrease or lack of disease symptoms following administration of the treatment or pharmacological agent. Other assays will be known to one of ordinary skill in the art and can be employed for measuring the level of the response. The amount of a treatment may be varied for example by increasing or decreasing the amount of a disclosed compound and/or pharmaceutical composition, by changing the disclosed compound and/or pharmaceutical composition administered, by changing the route of administration, by changing the dosage timing and so on. Dosage can vary, and can be administered in one or more dose administrations daily, for one or several days. Guidance can be found in the literature for appropriate dosages for given classes of pharmaceutical products.

As used herein, the term “prophylactically effective amount” refers to an amount effective for preventing onset or initiation of a disease or condition.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

The term “pharmaceutically acceptable” describes a material that is not biologically or otherwise undesirable, i.e., without causing an unacceptable level of undesirable biological effects or interacting in a deleterious manner.

The term “pharmaceutically acceptable salts”, as used herein, means salts of the active principal agents which are prepared with acids or bases that are tolerated by a biological system or tolerated by a subject or tolerated by a biological system and tolerated by a subject when administered in a therapeutically effective amount. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include, but are not limited to; sodium, potassium, calcium, ammonium, organic amino, magnesium salt, lithium salt, strontium salt or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include, but are not limited to; those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like.

The term “pharmaceutically acceptable ester” refers to esters of compounds of the present disclosure which hydrolyze in vivo and include those that break down readily in the human body to leave the parent compound or a salt thereof. Examples of pharmaceutically acceptable, non-toxic esters of the present disclosure include C 1-to-C 6 alkyl esters and C 5-to-C 7 cycloalkyl esters, although C 1-to-C 4 alkyl esters are preferred. Esters of disclosed compounds can be prepared according to conventional methods. Pharmaceutically acceptable esters can be appended onto hydroxy groups by reaction of the compound that contains the hydroxy group with acid and an alkylcarboxylic acid such as acetic acid, or with acid and an arylcarboxylic acid such as benzoic acid. In the case of compounds containing carboxylic acid groups, the pharmaceutically acceptable esters are prepared from compounds containing the carboxylic acid groups by reaction of the compound with base such as triethylamine and an alkyl halide, for example with methyl iodide, benzyl iodide, cyclopentyl iodide or alkyl triflate. They also can be prepared by reaction of the compound with an acid such as hydrochloric acid and an alcohol such as ethanol or methanol.

The term “pharmaceutically acceptable amide” refers to non-toxic amides of the present disclosure derived from ammonia, primary C 1-to-C 6 alkyl amines and secondary C 1-to-C 6 dialkyl amines. In the case of secondary amines, the amine can also be in the form of a 5- or 6-membered heterocycle containing one nitrogen atom. Amides derived from ammonia, C 1-to-C 3 alkyl primary amides and C 1-to-C 2 dialkyl secondary amides are preferred. Amides of disclosed compounds can be prepared according to conventional methods. Pharmaceutically acceptable amides can be prepared from compounds containing primary or secondary amine groups by reaction of the compound that contains the amino group with an alkyl anhydride, aryl anhydride, acyl halide, or aroyl halide. In the case of compounds containing carboxylic acid groups, the pharmaceutically acceptable amides are prepared from compounds containing the carboxylic acid groups by reaction of the compound with base such as triethylamine, a dehydrating agent such as dicyclohexyl carbodiimide or carbonyl diimidazole, and an alkyl amine, dialkylamine, for example with methylamine, diethylamine, and piperidine. They also can be prepared by reaction of the compound with an acid such as sulfuric acid and an alkylcarboxylic acid such as acetic acid, or with acid and an arylcarboxylic acid such as benzoic acid under dehydrating conditions such as with molecular sieves added. The composition can contain a compound of the present disclosure in the form of a pharmaceutically acceptable prodrug.

The term “pharmaceutically acceptable prodrug” or “prodrug” represents those prodrugs of the compounds of the present disclosure which are, within the scope of sound medical judgment, suitable for use in contact with the tissues of humans and lower animals without undue toxicity, irritation, allergic response, and the like, commensurate with a reasonable benefit/risk ratio, and effective for their intended use. Prodrugs of the present disclosure can be rapidly transformed in vivo to a parent compound having a structure of a disclosed compound, for example, by hydrolysis in blood. A thorough discussion is provided in T. Higuchi and V. Stella, Pro-drugs as Novel Delivery Systems, V. 14 of the A.C.S. Symposium Series, and in Edward B. Roche, ed., Bioreversible Carriers in Drug Design, American Pharmaceutical Association and Pergamon Press (1987).

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

The term “contacting” as used herein refers to bringing a disclosed compound or pharmaceutical composition in proximity to a cell, a target protein, or other biological entity together in such a manner that the disclosed compound or pharmaceutical composition can affect the activity of the a cell, target protein, or other biological entity, either directly; i.e., by interacting with the cell, target protein, or other biological entity itself, or indirectly; i.e., by interacting with another molecule, co-factor, factor, or protein on which the activity of the cell, target protein, or other biological entity itself is dependent.

Described herein are α-helix mimetics that have therapeutic or clinical utility. Also described herein are methods of synthesizing the α-helix mimetics Also described herein are methods of administering the α-helix mimetics to a subject in need thereof. In some aspects, the subject can have a cancer such as, for example, prostate cancer Other compositions, compounds, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

In various aspects, the disclosed compounds can possess at least one center of asymmetry, they can be present in the form of their racemates, in the form of the pure enantiomers and/or diastereomers or in the form of mixtures of these enantiomers and/or diastereomers. The stereoisomers can be present in the mixtures in any arbitrary proportions. In some aspects, provided this is possible, the disclosed compounds can be present in the form of the tautomers.

Thus, methods which are known per se can be used, for example, to separate the disclosed compounds which possess one or more chiral centers and occur as racemates into their optical isomers, i.e., enantiomers or diastereomers. The separation can be effected by means of column separation on chiral phases or by means of recrystallization from an optically active solvent or using an optically active acid or base or by means of derivatizing with an optically active reagent, such as an optically active alcohol, and subsequently cleaving off the residue.

In various aspects, the disclosed compounds can be in the form of a co-crystal. The term “co-crystal” means a physical association of two or more molecules which owe their stability through non-covalent interaction. One or more components of this molecular complex provide a stable framework in the crystalline lattice. In certain instances, the guest molecules are incorporated in the crystalline lattice as anhydrates or solvates, see e.g. “Crystal Engineering of the Composition of Pharmaceutical Phases. Do Pharmaceutical Co-crystals Represent a New Path to Improved Medicines?” Almarasson, O., et. al., The Royal Society of Chemistry, 1889-1896, 2004. Preferred co-crystals include p-toluenesulfonic acid and benzenesulfonic acid.

The term “pharmaceutically acceptable co-crystal” means one that is compatible with the other ingredients of the formulation and not deleterious to the recipient thereof.

In a further aspect, the disclosed compounds can be isolated as solvates and, in particular, as hydrates of a disclosed compound, which can be obtained, for example, by crystallization from a solvent or from aqueous solution. In this connection, one, two, three or any arbitrary number of solvate or water molecules can combine with the compounds according to the invention to form solvates and hydrates.

The disclosed compounds can be used in the form of salts derived from inorganic or organic acids. Pharmaceutically acceptable salts include salts of acidic or basic groups present in the disclosed compounds. Suitable pharmaceutically acceptable salts include base addition salts, including alkali metal salts, e.g., sodium or potassium salts; alkaline earth metal salts, e.g., calcium or magnesium salts; and salts formed with suitable organic ligands, e.g., quaternary ammonium salts, which may be similarly prepared by reacting the drug compound with a suitable pharmaceutically acceptable base. The salts can be prepared in situ during the final isolation and purification of the compounds of the present disclosure; or following final isolation by reacting a free base function, such as a secondary or tertiary amine, of a disclosed compound with a suitable inorganic or organic acid; or reacting a free acid function, such as a carboxylic acid, of a disclosed compound with a suitable inorganic or organic base.

Acidic addition salts can be prepared in situ during the final isolation and purification of a disclosed compound, or separately by reacting moieties comprising one or more nitrogen groups with a suitable acid. In various aspects, acids which may be employed to form pharmaceutically acceptable acid addition salts include such inorganic acids as hydrochloric acid, sulfuric acid and phosphoric acid and such organic acids as oxalic acid, maleic acid, succinic acid and citric acid. In a further aspect, salts further include, but are not limited, to the following: hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate, butyrate, camphorate, camphorsulfonate, digluconate, glycerophosphate, hemisulfate, heptanoate, hexanoate, fumarate, hydrochloride, 2-hydroxyethanesulfonate (isethionate), nicotinate, 2-naphthalenesulfonate, oxalate, pectinate, persulfate, 3-phenylpropionate, picrate, pivalate, propionate, succinate, tartrate, thiocyanate, phosphate, glutamate, bicarbonate, undecanoate, and pamoate (i.e., 1,1′-methylene-bis-(2-hydroxy-3-naphthoate)) salts. Also, basic nitrogen-containing groups can be quaternized with such agents as lower alkyl halides, such as methyl, ethyl, propyl, and butyl chloride, bromides, and iodides; dialkyl sulfates like dimethyl, diethyl, dibutyl, and diamyl sulfates, long chain halides such as decyl, lauryl, myristyl and stearyl chlorides, bromides and iodides, aralkyl halides like benzyl and phenethyl bromides, and others.

Basic addition salts can be prepared in situ during the final isolation and purification of a disclosed compound, or separately by reacting carboxylic acid moieties with a suitable base such as the hydroxide, carbonate or bicarbonate of a pharmaceutical acceptable metal cation or with ammonia, or an organic primary, secondary or tertiary amine. Pharmaceutical acceptable salts include, but are not limited to, cations based on the alkali and alkaline earth metals, such as sodium, lithium, potassium, calcium, magnesium, aluminum salts and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. Other representative organic amines useful for the formation of base addition salts include diethylamine, ethylenediamine, ethanolamine, diethanolamine, piperazine and the like. In further aspects, bases which may be used in the preparation of pharmaceutically acceptable salts include the following: ammonia, L-arginine, benethamine, benzathine, calcium hydroxide, choline, deanol, diethanolamine, diethylamine, 2-(diethylamino)-ethanol, ethanolamine, ethylenediamine, N-methyl-glucamine, hydrabamine, 1H-imidazole, L-lysine, magnesium hydroxide, 4-(2-hydroxyethyl)-morpholine, piperazine, potassium hydroxide, 1-(2-hydroxyethyl)-pyrrolidine, secondary amine, sodium hydroxide, triethanolamine, tromethamine and zinc hydroxide.

In various aspects, the present disclosure relates to pharmaceutical compositions comprising a therapeutically effective amount of at least one disclosed compound, at least one product of a disclosed method, or a pharmaceutically acceptable salt thereof. As used herein, “pharmaceutically-acceptable carriers” means one or more of a pharmaceutically acceptable diluents, preservatives, antioxidants, solubilizers, emulsifiers, coloring agents, releasing agents, coating agents, sweetening, flavoring and perfuming agents, and adjuvants. The disclosed pharmaceutical compositions can be conveniently presented in unit dosage form and prepared by any of the methods well known in the art of pharmacy and pharmaceutical sciences.

In a further aspect, the disclosed pharmaceutical compositions comprise a therapeutically effective amount of at least one disclosed compound, at least one product of a disclosed method, or a pharmaceutically acceptable salt thereof as an active ingredient, a pharmaceutically acceptable carrier, optionally one or more other therapeutic agent, and optionally one or more adjuvant. The disclosed pharmaceutical compositions include those suitable for oral, rectal, topical, pulmonary, nasal, and parenteral administration, although the most suitable route in any given case will depend on the particular host, and nature and severity of the conditions for which the active ingredient is being administered. In a further aspect, the disclosed pharmaceutical composition can be formulated to allow administration orally, nasally, via inhalation, parenterally, paracancerally, transmucosally, transdermally, intramuscularly, intravenously, intradermally, subcutaneously, intraperitoneally, intraventricularly, intracranially and intratumorally.

As used herein, “parenteral administration” includes administration by bolus injection or infusion, as well as administration by intravenous, intramuscular, intraarterial, intrathecal, intracapsular, intraorbital, intracardiac, intradermal, intraperitoneal, transtracheal, subcutaneous, subcuticular, intraarticular, subcapsular subarachnoid, intraspinal, epidural and intrasternal injection and infusion.

In various aspects, the present disclosure also relates to a pharmaceutical composition comprising a pharmaceutically acceptable carrier or diluent and, as active ingredient, a therapeutically effective amount of a disclosed compound, a product of a disclosed method of making, a pharmaceutically acceptable salt, a hydrate thereof, a solvate thereof, a polymorph thereof, or a stereochemically isomeric form thereof. In a further aspect, a disclosed compound, a product of a disclosed method of making, a pharmaceutically acceptable salt, a hydrate thereof, a solvate thereof, a polymorph thereof, or a stereochemically isomeric form thereof, or any subgroup or combination thereof may be formulated into various pharmaceutical forms for administration purposes.

Pharmaceutically acceptable salts can be prepared from pharmaceutically acceptable non-toxic bases or acids. For therapeutic use, salts of the disclosed compounds are those wherein the counter ion is pharmaceutically acceptable. However, salts of acids and bases which are non-pharmaceutically acceptable may also find use, for example, in the preparation or purification of a pharmaceutically acceptable compound. All salts, whether pharmaceutically acceptable or not, are contemplated by the present disclosure. Pharmaceutically acceptable acid and base addition salts are meant to comprise the therapeutically active non-toxic acid and base addition salt forms which the disclosed compounds are able to form.

In various aspects, a disclosed compound comprising an acidic group or moiety, e.g., a carboxylic acid group, can be used to prepare a pharmaceutically acceptable salt. For example, such a disclosed compound may comprise an isolation step comprising treatment with a suitable inorganic or organic base. In some cases, it may be desirable in practice to initially isolate a compound from the reaction mixture as a pharmaceutically unacceptable salt and then simply convert the latter back to the free acid compound by treatment with an acidic reagent, and subsequently convert the free acid to a pharmaceutically acceptable base addition salt. These base addition salts can be readily prepared using conventional techniques, e.g., by treating the corresponding acidic compounds with an aqueous solution containing the desired pharmacologically acceptable cations and then evaporating the resulting solution to dryness, preferably under reduced pressure. Alternatively, they also can be prepared by mixing lower alkanolic solutions of the acidic compounds and the desired alkali metal alkoxide together, and then evaporating the resulting solution to dryness in the same manner as before.

Bases which can be used to prepare the pharmaceutically acceptable base-addition salts of the base compounds are those which can form non-toxic base-addition salts, i.e., salts containing pharmacologically acceptable cations such as, alkali metal cations (e.g., lithium, potassium and sodium), alkaline earth metal cations (e.g., calcium and magnesium), ammonium or other water-soluble amine addition salts such as N-methylglucamine-(meglumine), lower alkanolammonium and other such bases of organic amines. In a further aspect, derived from pharmaceutically acceptable organic non-toxic bases include primary, secondary, and tertiary amines, as well as cyclic amines and substituted amines such as naturally occurring and synthesized substituted amines. In various aspects, such pharmaceutically acceptable organic non-toxic bases include, but are not limited to, ammonia, methylamine, ethylamine, propylamine, isopropylamine, any of the four butylamine isomers, betaine, caffeine, choline, dimethylamine, diethylamine, diethanolamine, dipropylamine, diisopropylamine, di-n-butylamine, N,N′-dibenzylethylenediamine, pyrrolidine, piperidine, morpholine, trimethylamine, triethylamine, tripropylamine, tromethamine, 2-diethylaminoethanol, 2-dimethylaminoethanol, ethanolamine, quinuclidine, pyridine, quinoline and isoquinoline; benzathine, N-methyl-D-glucamine, ethylenediamine, N-ethylmorpholine, N-ethylpiperidine, glucamine, glucosamine, methylglucamine, morpholine, piperazine, piperidine, polyamine resins, procaine, purines, theobromine, hydrabamine salts, and salts with amino acids such as, for example, histidine, arginine, lysine and the like. The foregoing salt forms can be converted by treatment with acid back into the free acid form.

In various aspects, a disclosed compound comprising a protonatable group or moiety, e.g., an amino group, can be used to prepare a pharmaceutically acceptable salt. For example, such a disclosed compound may comprise an isolation step comprising treatment with a suitable inorganic or organic acid. In some cases, it may be desirable in practice to initially isolate a compound from the reaction mixture as a pharmaceutically unacceptable salt and then simply convert the latter back to the free base compound by treatment with a basic reagent, and subsequently convert the free base to a pharmaceutically acceptable acid addition salt. These acid addition salts can be readily prepared using conventional techniques, e.g., by treating the corresponding basic compounds with an aqueous solution containing the desired pharmacologically acceptable anions and then evaporating the resulting solution to dryness, preferably under reduced pressure. Alternatively, they also can be prepared by treating the free base form of the disclosed compound with a suitable pharmaceutically acceptable non-toxic inorganic or organic acid.

Acids that can be used to prepare the pharmaceutically acceptable acid-addition salts of the base compounds are those which can form non-toxic acid-addition salts, i.e., salts containing pharmacologically acceptable anions formed from their corresponding inorganic and organic acids. Exemplary, but non-limiting, inorganic acids include hydrochloric hydrobromic, sulfuric, nitric, phosphoric and the like. Exemplary, but non-limiting, organic acids include acetic, benzenesulfonic, benzoic, camphorsulfonic, citric, ethanesulfonic, fumaric, gluconic, glutamic, isethionic, lactic, maleic, malic, mandelicmethanesulfonic, mucic, pamoic, pantothenic, succinic, tartaric, p-toluenesulfonic acid and the like. In a further aspect, the acid-addition salt comprises an anion formed from hydrobromic, hydrochloric, maleic, phosphoric, sulfuric, and tartaric acids.

In practice, the compounds of the present disclosure, or pharmaceutically acceptable salts thereof, of the present disclosure can be combined as the active ingredient in intimate admixture with a pharmaceutical carrier according to conventional pharmaceutical compounding techniques. The carrier can take a wide variety of forms depending on the form of preparation desired for administration, e.g., oral or parenteral (including intravenous). Thus, the pharmaceutical compositions of the present disclosure can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compounds of the present disclosure, and/or pharmaceutically acceptable salt(s) thereof, can also be administered by controlled release means and/or delivery devices. The compositions can be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

It is especially advantageous to formulate the aforementioned pharmaceutical compositions in unit dosage form for ease of administration and uniformity of dosage. The term “unit dosage form,” as used herein, refers to physically discrete units suitable as unitary dosages, each unit containing a predetermined quantity of active ingredient calculated to produce the desired therapeutic effect in association with the required pharmaceutical carrier. That is, a “unit dosage form” is taken to mean a single dose wherein all active and inactive ingredients are combined in a suitable system, such that the patient or person administering the drug to the patient can open a single container or package with the entire dose contained therein, and does not have to mix any components together from two or more containers or packages. Typical examples of unit dosage forms are tablets (including scored or coated tablets), capsules or pills for oral administration; single dose vials for injectable solutions or suspension; suppositories for rectal administration; powder packets; wafers; and segregated multiples thereof. This list of unit dosage forms is not intended to be limiting in any way, but merely to represent typical examples of unit dosage forms.

The pharmaceutical compositions disclosed herein comprise a compound of the present disclosure (or pharmaceutically acceptable salts thereof) as an active ingredient, a pharmaceutically acceptable carrier, and optionally one or more additional therapeutic agents. In various aspects, the disclosed pharmaceutical compositions can include a pharmaceutically acceptable carrier and a disclosed compound, or a pharmaceutically acceptable salt thereof. In a further aspect, a disclosed compound, or pharmaceutically acceptable salt thereof, can also be included in a pharmaceutical composition in combination with one or more other therapeutically active compounds. The instant compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

Techniques and compositions for making dosage forms useful for materials and methods described herein are described, for example, in the following references: Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol 40 (Gilbert S. Banker, Christopher T. Rhodes, Eds.).

The compounds described herein are typically to be administered in admixture with suitable pharmaceutical diluents, excipients, extenders, or carriers (termed herein as a pharmaceutically acceptable carrier, or a carrier) suitably selected with respect to the intended form of administration and as consistent with conventional pharmaceutical practices. The deliverable compound will be in a form suitable for oral, rectal, topical, intravenous injection or parenteral administration. Carriers include solids or liquids, and the type of carrier is chosen based on the type of administration being used. The compounds may be administered as a dosage that has a known quantity of the compound.

Because of the ease in administration, oral administration can be a preferred dosage form, and tablets and capsules represent the most advantageous oral dosage unit forms in which case solid pharmaceutical carriers are obviously employed. However, other dosage forms may be suitable depending upon clinical population (e.g., age and severity of clinical condition), solubility properties of the specific disclosed compound used, and the like. Accordingly, the disclosed compounds can be used in oral dosage forms such as pills, powders, granules, elixirs, tinctures, suspensions, syrups, and emulsions. In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques.

The disclosed pharmaceutical compositions in an oral dosage form can comprise one or more pharmaceutical excipient and/or additive. Non-limiting examples of suitable excipients and additives include gelatin, natural sugars such as raw sugar or lactose, lecithin, pectin, starches (for example corn starch or amylose), dextran, polyvinyl pyrrolidone, polyvinyl acetate, gum arabic, alginic acid, tylose, talcum, lycopodium, silica gel (for example colloidal), cellulose, cellulose derivatives (for example cellulose ethers in which the cellulose hydroxy groups are partially etherified with lower saturated aliphatic alcohols and/or lower saturated, aliphatic oxyalcohols, for example methyl oxypropyl cellulose, methyl cellulose, hydroxypropyl methyl cellulose, hydroxypropyl methyl cellulose phthalate), fatty acids as well as magnesium, calcium or aluminum salts of fatty acids with 12 to 22 carbon atoms, in particular saturated (for example stearates), emulsifiers, oils and fats, in particular vegetable (for example, peanut oil, castor oil, olive oil, sesame oil, cottonseed oil, corn oil, wheat germ oil, sunflower seed oil, cod liver oil, in each case also optionally hydrated); glycerol esters and polyglycerol esters of saturated fatty acids C₁₂H₂₄O₂ to C₁₈H₃₆O₂ and their mixtures, it being possible for the glycerol hydroxy groups to be totally or also only partly esterified (for example mono-, di- and triglycerides); pharmaceutically acceptable mono- or multivalent alcohols and polyglycols such as polyethylene glycol and derivatives thereof, esters of aliphatic saturated or unsaturated fatty acids (2 to 22 carbon atoms, in particular 10-18 carbon atoms) with monovalent aliphatic alcohols (1 to 20 carbon atoms) or multivalent alcohols such as glycols, glycerol, diethylene glycol, pentacrythritol, sorbitol, mannitol and the like, which may optionally also be etherified, esters of citric acid with primary alcohols, acetic acid, urea, benzyl benzoate, dioxolanes, glyceroformals, tetrahydrofurfuryl alcohol, polyglycol ethers with C1-C12-alcohols, dimethylacetamide, lactamides, lactates, ethylcarbonates, silicones (in particular medium-viscous polydimethyl siloxanes), calcium carbonate, sodium carbonate, calcium phosphate, sodium phosphate, magnesium carbonate and the like.

Other auxiliary substances useful in preparing an oral dosage form are those which cause disintegration (so-called disintegrants), such as: cross-linked polyvinyl pyrrolidone, sodium carboxymethyl starch, sodium carboxymethyl cellulose or microcrystalline cellulose. Conventional coating substances may also be used to produce the oral dosage form. Those that may for example be considered are: polymerizates as well as copolymerizates of acrylic acid and/or methacrylic acid and/or their esters; copolymerizates of acrylic and methacrylic acid esters with a lower ammonium group content (for example EudragitR RS), copolymerizates of acrylic and methacrylic acid esters and trimethyl ammonium methacrylate (for example EudragitR RL); polyvinyl acetate; fats, oils, waxes, fatty alcohols; hydroxypropyl methyl cellulose phthalate or acetate succinate; cellulose acetate phthalate, starch acetate phthalate as well as polyvinyl acetate phthalate, carboxy methyl cellulose; methyl cellulose phthalate, methyl cellulose succinate, -phthalate succinate as well as methyl cellulose phthalic acid half ester; zein; ethyl cellulose as well as ethyl cellulose succinate; shellac, gluten; ethylcarboxyethyl cellulose; ethacrylate-maleic acid anhydride copolymer; maleic acid anhydride-vinyl methyl ether copolymer; styrol-maleic acid copolymerizate; 2-ethyl-hexyl-acrylate maleic acid anhydride; crotonic acid-vinyl acetate copolymer; glutaminic acid/glutamic acid ester copolymer; carboxymethylethylcellulose glycerol monooctanoate; cellulose acetate succinate; polyarginine.

Plasticizing agents that may be considered as coating substances in the disclosed oral dosage forms are: citric and tartaric acid esters (acetyl-triethyl citrate, acetyl tributyl-, tributyl-, triethyl-citrate); glycerol and glycerol esters (glycerol diacetate, -triacetate, acetylated monoglycerides, castor oil); phthalic acid esters (dibutyl-, diamyl-, diethyl-, dimethyl-, dipropyl-phthalate), di-(2-methoxy- or 2-ethoxyethyl)-phthalate, ethylphthalyl glycolate, butylphthalylethyl glycolate and butylglycolate; alcohols (propylene glycol, polyethylene glycol of various chain lengths), adipates (diethyladipate, di-(2-methoxy- or 2-ethoxyethyl)-adipate; benzophenone; diethyl- and diburylsebacate, dibutylsuccinate, dibutyltartrate; diethylene glycol dipropionate; ethyleneglycol diacetate, -dibutyrate, -dipropionate; tributyl phosphate, tributyrin; polyethylene glycol sorbitan monooleate (polysorbates such as Polysorbar 50); sorbitan monooleate.

Moreover, suitable binders, lubricants, disintegrating agents, coloring agents, flavoring agents, flow-inducing agents, and melting agents may be included as carriers. The pharmaceutical carrier employed can be, for example, a solid, liquid, or gas. Examples of solid carriers include, but are not limited to, lactose, terra alba, sucrose, glucose, methylcellulose, dicalcium phosphate, calcium sulfate, mannitol, sorbitol talc, starch, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In various aspects, a binder can include, for example, starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. In a further aspect, a disintegrator can include, for example, starch, methyl cellulose, agar, bentonite, xanthan gum, and the like.

In various aspects, an oral dosage form, such as a solid dosage form, can comprise a disclosed compound that is attached to polymers as targetable drug carriers or as a prodrug. Suitable biodegradable polymers useful in achieving controlled release of a drug include, for example, polylactic acid, polyglycolic acid, copolymers of polylactic and polyglycolic acid, caprolactones, polyhydroxy butyric acid, polyorthoesters, polyacetals, polydihydropyrans, polycyanoacylates, and hydrogels, preferably covalently crosslinked hydrogels.

Tablets may contain the active ingredient in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period.

A tablet containing a disclosed compound can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

In various aspects, a solid oral dosage form, such as a tablet, can be coated with an enteric coating to prevent ready decomposition in the stomach. In various aspects, enteric coating agents include, but are not limited to, hydroxypropylmethylcellulose phthalate, methacrylic acid-methacrylic acid ester copolymer, polyvinyl acetate-phthalate and cellulose acetate phthalate. Akihiko Hasegawa “Application of solid dispersions of Nifedipine with enteric coating agent to prepare a sustained-release dosage form” Chem. Pharm. Bull. 33:1615-1619 (1985). Various enteric coating materials may be selected on the basis of testing to achieve an enteric coated dosage form designed ab initio to have a preferable combination of dissolution time, coating thicknesses and diametral crushing strength (e.g., see S. C. Porter et al. “The Properties of Enteric Tablet Coatings Made From Polyvinyl Acetate-phthalate and Cellulose acetate Phthalate”, J. Pharm. Pharmacol. 22:42p (1970)). In a further aspect, the enteric coating may comprise hydroxypropyl-methylcellulose phthalate, methacrylic acid-methacrylic acid ester copolymer, polyvinyl acetate-phthalate and cellulose acetate phthalate.

In various aspects, an oral dosage form can be a solid dispersion with a water soluble or a water insoluble carrier. Examples of water soluble or water insoluble carrier include, but are not limited to, polyethylene glycol, polyvinylpyrrolidone, hydroxypropylmethyl-cellulose, phosphatidylcholine, polyoxyethylene hydrogenated castor oil, hydroxypropylmethylcellulose phthalate, carboxymethylethylcellulose, or hydroxypropylmethylcellulose, ethyl cellulose, or stearic acid.

In various aspects, an oral dosage form can be in a liquid dosage form, including those that are ingested, or alternatively, administered as a mouth wash or gargle. For example, a liquid dosage form can include aqueous suspensions, which contain the active materials in admixture with excipients suitable for the manufacture of aqueous suspensions. In addition, oily suspensions may be formulated by suspending the active ingredient in a vegetable oil, for example arachis oil, olive oil, sesame oil or coconut oil, or in a mineral oil such as liquid paraffin. Oily suspensions may also contain various excipients. The pharmaceutical compositions of the present disclosure may also be in the form of oil-in-water emulsions, which may also contain excipients such as sweetening and flavoring agents.

For the preparation of solutions or suspensions it is, for example, possible to use water, particularly sterile water, or physiologically acceptable organic solvents, such as alcohols (ethanol, propanol, isopropanol, 1,2-propylene glycol, polyglycols and their derivatives, fatty alcohols, partial esters of glycerol), oils (for example peanut oil, olive oil, sesame oil, almond oil, sunflower oil, soya bean oil, castor oil, bovine hoof oil), paraffins, dimethyl sulfoxide, triglycerides and the like.

In the case of a liquid dosage form such as a drinkable solutions, the following substances may be used as stabilizers or solubilizers: lower aliphatic mono- and multivalent alcohols with 2-4 carbon atoms, such as ethanol, n-propanol, glycerol, polyethylene glycols with molecular weights between 200-600 (for example 1 to 40% aqueous solution), diethylene glycol monoethyl ether, 1,2-propylene glycol, organic amides, for example amides of aliphatic C1-C6-carboxylic acids with ammonia or primary, secondary or tertiary C1-C4-amines or C1-C4-hydroxy amines such as urea, urethane, acetamide, N-methyl acetamide, N,N-diethyl acetamide, N,N-dimethyl acetamide, lower aliphatic amines and diamines with 2-6 carbon atoms, such as ethylene diamine, hydroxyethyl theophylline, tromethamine (for example as 0.1 to 20% aqueous solution), aliphatic amino acids.

In preparing the disclosed liquid dosage form can comprise solubilizers and emulsifiers such as the following non-limiting examples can be used: polyvinyl pyrrolidone, sorbitan fatty acid esters such as sorbitan trioleate, phosphatides such as lecithin, acacia, tragacanth, polyoxyethylated sorbitan monooleate and other ethoxylated fatty acid esters of sorbitan, polyoxyethylated fats, polyoxyethylated oleotriglycerides, Iinolizated oleotriglycerides, polyethylene oxide condensation products of fatty alcohols, alkylphenols or fatty acids or also 1-methyl-3-(2-hydroxyethyl)imidazolidone-(2). In this context, polyoxyethylated means that the substances in question contain polyoxyethylene chains, the degree of polymerization of which generally lies between 2 and 40 and in particular between 10 and 20. Polyoxyethylated substances of this kind may for example be obtained by reaction of hydroxyl group-containing compounds (for example mono- or diglycerides or unsaturated compounds such as those containing oleic acid radicals) with ethylene oxide (for example 40 Mol ethylene oxide per 1 Mol glyceride). Examples of oleotriglycerides are olive oil, peanut oil, castor oil, sesame oil, cottonseed oil, corn oil. See also Dr. H. P. Fiedler “Lexikon der Hillsstoffe für Pharmazie, Kostnetik und angrenzende Gebiete” 1971, pages 191-195.

In various aspects, a liquid dosage form can further comprise preservatives, stabilizers, buffer substances, flavor correcting agents, sweeteners, colorants, antioxidants and complex formers and the like. Complex formers which may be for example be considered are: chelate formers such as ethylene diamine retrascetic acid, nitrilotriacetic acid, diethylene triamine pentacetic acid and their salts.

It may optionally be necessary to stabilize a liquid dosage form with physiologically acceptable bases or buffers to a pH range of approximately 6 to 9. Preference may be given to as neutral or weakly basic a pH value as possible (up to pH 8).

In order to enhance the solubility and/or the stability of a disclosed compound in a disclosed liquid dosage form, a parenteral injection form, or an intravenous injectable form, it can be advantageous to employ α-, β- or γ-cyclodextrins or their derivatives, in particular hydroxyalkyl substituted cyclodextrins, e.g. 2-hydroxypropyl-β-cyclodextrin or sulfobutyl-β-cyclodextrin. Also co-solvents such as alcohols may improve the solubility and/or the stability of the compounds according to the present disclosure in pharmaceutical compositions.

In various aspects, a disclosed liquid dosage form, a parenteral injection form, or an intravenous injectable form can further comprise liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

Pharmaceutical compositions of the present disclosure suitable injection, such as parenteral administration, such as intravenous, intramuscular, or subcutaneous administration. Pharmaceutical compositions for injection can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present disclosure suitable for parenteral administration can include sterile aqueous or oleaginous solutions, suspensions, or dispersions. Furthermore, the compositions can be in the form of sterile powders for the extemporaneous preparation of such sterile injectable solutions or dispersions. In some aspects, the final injectable form is sterile and must be effectively fluid for use in a syringe. The pharmaceutical compositions should be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Injectable solutions, for example, can be prepared in which the carrier comprises saline solution, glucose solution or a mixture of saline and glucose solution. Injectable suspensions may also be prepared in which case appropriate liquid carriers, suspending agents and the like may be employed. In some aspects, a disclosed parenteral formulation can comprise about 0.01-0.1 M, e.g. about 0.05 M, phosphate buffer. In a further aspect, a disclosed parenteral formulation can comprise about 0.9% saline.

In various aspects, a disclosed parenteral pharmaceutical composition can comprise pharmaceutically acceptable carriers such as aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include but not limited to water, alcoholic/aqueous solutions, emulsions, or suspensions, including saline and buffered media. Parenteral vehicles can include mannitol, normal serum albumin, sodium chloride solution, Ringer's dextrose, dextrose and sodium chloride, lactated Ringer's, and fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, chelating agents, inert gases, and the like. In a further aspect, a disclosed parenteral pharmaceutical composition can comprise may contain minor amounts of additives such as substances that enhance isotonicity and chemical stability, e.g., buffers and preservatives. Also contemplated for injectable pharmaceutical compositions are solid form preparations that are intended to be converted, shortly before use, to liquid form preparations. Furthermore, other adjuvants can be included to render the formulation isotonic with the blood of the subject or patient.

The exact dosage and frequency of administration depends on the particular disclosed compound, a product of a disclosed method of making, a pharmaceutically acceptable salt, solvate, or polymorph thereof, a hydrate thereof, a solvate thereof, a polymorph thereof, or a stereochemically isomeric form thereof; the particular condition being treated and the severity of the condition being treated; various factors specific to the medical history of the subject to whom the dosage is administered such as the age; weight, sex, extent of disorder and general physical condition of the particular subject, as well as other medication the individual may be taking; as is well known to those skilled in the art. Furthermore, it is evident that said effective daily amount may be lowered or increased depending on the response of the treated subject and/or depending on the evaluation of the physician prescribing the compounds of the present disclosure.

Depending on the mode of administration, the pharmaceutical composition will comprise from 0.05 to 99% by weight, preferably from 0.1 to 70% by weight, more preferably from 0.1 to 50% by weight of the active ingredient, and, from 1 to 99.95% by weight, preferably from 30 to 99.9% by weight, more preferably from 50 to 99.9% by weight of a pharmaceutically acceptable carrier, all percentages being based on the total weight of the composition.

Sn appropriate dosage level can be from about 0.01 to 1000 mg per kg patient body weight per day and can be administered in single or multiple doses. In various aspects, the dosage level will be about 0.1 to about 500 mg/kg per day, about 0.1 to 250 mg/kg per day, or about 0.5 to 100 mg/kg per day. A suitable dosage level can be about 0.01 to 1000 mg/kg per day, about 0.01 to 500 mg/kg per day, about 0.01 to 250 mg/kg per day, about 0.05 to 100 mg/kg per day, or about 0.1 to 50 mg/kg per day. Within this range the dosage can be 0.05 to 0.5, 0.5 to 5.0 or 5.0 to 50 mg/kg per day. For oral administration, the compositions are preferably provided in the form of tablets containing 1.0 to 1000 mg of the active ingredient, particularly 1.0, 5.0, 10, 15, 20, 25, 50, 75, 100, 150, 200, 250, 300, 400, 500, 600, 750, 800, 900 and 1000 mg of the active ingredient for the symptomatic adjustment of the dosage of the patient to be treated. The compound can be administered on a regimen of 1 to 4 times per day, preferably once or twice per day. This dosing regimen can be adjusted to provide the optimal therapeutic response.

Such unit doses as described hereinabove and hereinafter can be administered more than once a day, for example, 2, 3, 4, 5 or 6 times a day. In various aspects, such unit doses can be administered 1 or 2 times per day, so that the total dosage for a 70 kg adult is in the range of 0.001 to about 15 mg per kg weight of subject per administration. In a further aspect, dosage is 0.01 to about 1.5 mg per kg weight of subject per administration, and such therapy can extend for a number of weeks or months, and in some cases, years. It will be understood, however, that the specific dose level for any particular patient will depend on a variety of factors including the activity of the specific compound employed; the age, body weight, general health, sex and diet of the individual being treated; the time and route of administration; the rate of excretion; other drugs that have previously been administered; and the severity of the particular disease undergoing therapy, as is well understood by those of skill in the area.

A typical dosage can be one 1 mg to about 100 mg tablet or 1 mg to about 300 mg taken once a day, or multiple times per day, or one time-release capsule or tablet taken once a day and containing a proportionally higher content of active ingredient. The time-release effect can be obtained by capsule materials that dissolve at different pH values, by capsules that release slowly by osmotic pressure, or by any other known means of controlled release.

It can be necessary to use dosages outside these ranges in some cases as will be apparent to those skilled in the art. Further, it is noted that the clinician or treating physician will know how and when to start, interrupt, adjust, or terminate therapy in conjunction with individual patient response.

The disclosed pharmaceutical compositions can further comprise other therapeutically active compounds, which are usually applied in the treatment of the above mentioned pathological or clinical conditions.

It is understood that the disclosed compositions can be prepared from the disclosed compounds. It is also understood that the disclosed compositions can be employed in the disclosed methods of using.

As already mentioned, the present disclosure relates to a pharmaceutical composition comprising a therapeutically effective amount of a disclosed compound, a product of a disclosed method of making, a pharmaceutically acceptable salt, a hydrate thereof, a solvate thereof, a polymorph thereof, and a pharmaceutically acceptable carrier. Additionally, the present disclosure relates to a process for preparing such a pharmaceutical composition, characterized in that a pharmaceutically acceptable carrier is intimately mixed with a therapeutically effective amount of a compound according to the present disclosure.

As already mentioned, the present disclosure also relates to a pharmaceutical composition comprising a disclosed compound, a product of a disclosed method of making, a pharmaceutically acceptable salt, a hydrate thereof, a solvate thereof, a polymorph thereof, and one or more other drugs in the treatment, prevention, control, amelioration, or reduction of risk of diseases or conditions for a disclosed compound or the other drugs may have utility as well as to the use of such a composition for the manufacture of a medicament. The present disclosure also relates to a combination of disclosed compound, a product of a disclosed method of making, a pharmaceutically acceptable salt, a hydrate thereof, a solvate thereof, a polymorph thereof, and an inhibitor of an anti-apoptotic protein such as a disclosed compound. The present disclosure also relates to such a combination for use as a medicine. The present disclosure also relates to a product comprising (a) disclosed compound, a product of a disclosed method of making, a pharmaceutically acceptable salt, a hydrate thereof, a solvate thereof, a polymorph thereof, and (b) an additional anticancer therapeutic agent, as a combined preparation for simultaneous, separate or sequential use in the treatment or prevention of a condition in a mammal, including a human, the treatment or prevention of which is affected or facilitated by the modulatory effect of the disclosed compound and the additional therapeutic agent. The different drugs of such a combination or product may be combined in a single preparation together with pharmaceutically acceptable carriers or diluents, or they may each be present in a separate preparation together with pharmaceutically acceptable carriers or diluents.

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### Aspects

The present disclosure can be described in accordance with the following numbered aspects, which should not be confused with the claims.

Aspect 1. A compound having Formula I or a pharmaceutically acceptable salt thereof

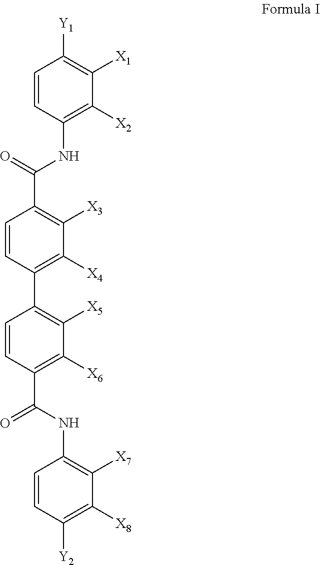

wherein each of X₁-X₈ is individually selected from H, OR₁, NR₁R₂, SR₁, or PR₁R₂;
wherein each of Y₁-Y₂ is individually selected from H, CO₂R₁, C(═O)NR₁R₂, C(═O)R₁, OR₁, NR₁R₂, SR₁, or PR₁R₂;
wherein each of R₁ and R₂ is individually selected from H, linear or branched C1-C20 alkyl, C3-C20 aryl, linear or branched C2-C20 alkenyl, linear or branched C2-C20 alkynyl, or C3-C20 arylalkyl; and
wherein when R₁, R₂, or both R₁ and R₂ are linear or branched C1-C20 alkyl, C3-C20 aryl, linear or branched C2-C20 alkenyl, linear or branched C2-C20 alkynyl, or C3-C20 arylalkyl, R₁, R₂, or both R₁ and R₂ can be optionally substituted with an ether, amine, alkoxy, ester, amide, thiol, phosphine, or any combination thereof.

Aspect 2. The compound or pharmaceutically acceptable salt of aspect 1, wherein at least four of X₁-X₈ are H.

Aspect 3. The compound or pharmaceutically acceptable salt of aspect 1 or 2, wherein X₁. X₄, X₅, and Xₐ are H.

Aspect 4. The compound or pharmaceutically acceptable salt of aspect 1 or 2, wherein X₁. X₄, X₆, and Xₐ are H.

Aspect 5. The compound or pharmaceutically acceptable salt of aspect 1 or 2, wherein X₂. X₄, X₆, and Xₐ are H.

Aspect 6. The compound or pharmaceutically acceptable salt of aspect 1 or 2, wherein X₁. X₃, X₅, and Xₐ are H.

Aspect 7. The compound or pharmaceutically acceptable salt of aspect 1 or 2, wherein X₁. X₃, X₅, and X₇ are H.

Aspect 8. The compound or pharmaceutically acceptable salt of aspect 1 or 2, wherein X₂. X₄, X₅, and Xₐ are H.

Aspect 9. The compound or pharmaceutically acceptable salt of aspect 1 or 2, wherein X₂. X₄, X₅, and X₇ are H.

Aspect 10. The compound or pharmaceutically acceptable salt of aspect 1 or 2, wherein X₁. X₄, X₅, and X₇ are H.

Aspect 11. The compound or pharmaceutically acceptable salt of any one of aspects 1-10, wherein at least four of X₁-X₈ are OR₁.

Aspect 12. The compound or pharmaceutically acceptable salt of aspect 1 or 2, wherein X₂. X₃, X₆, and X₇ are OR₁.

Aspect 13. The compound or pharmaceutically acceptable salt of aspect 1 or 2, wherein X₂. X₃, X₅, and X₇ are OR₁.

Aspect 14. The compound or pharmaceutically acceptable salt of aspect 1 or 2, wherein X₁. X₃, X₅, and X₇ are OR₁.

Aspect 15. The compound or pharmaceutically acceptable salt of aspect 1 or 2, wherein X₂. X₄, X₆, and X₇ are OR₁.

Aspect 16. The compound or pharmaceutically acceptable salt of aspect 1 or 2, wherein X₂. X₄, X₆, and X₈ are OR₁.

Aspect 17. The compound or pharmaceutically acceptable salt of aspect 1 or 2, wherein X₁. X₃, X₆, and X₇ are OR₁.

Aspect 18. The compound or pharmaceutically acceptable salt of aspect 1 or 2, wherein X₁. X₃, X₆, and X₈ are OR₁.

Aspect 19. The compound or pharmaceutically acceptable salt of aspect 1 or 2, wherein X₂. X₃, X₆, and X₈ are OR₁.

Aspect 20. The compound or pharmaceutically acceptable salt of any one of aspects 1-19, wherein Y₁ and Y₂ are CO₂H.

Aspect 21. The compound or pharmaceutically acceptable salt of any one of aspects 1-19, wherein Y₁ is H and Y₂ is CO₂H.

Aspect 22. The compound or pharmaceutically acceptable salt of any one of aspects 1-19, wherein Y₁ is CO₂H and Y₂ is H.

Aspect 23. The compound or pharmaceutically acceptable salt of any one of aspects 11-22, wherein each R₁ is individually selected from sec-butyl, isobutyl, isopropyl, ethyl, isopentyl, methylbutyl, n-pentyl, phenylethyl, benzyl, naphthyl, or n-butyl.

Aspect 24. The compound or pharmaceutically acceptable salt of any one of aspects 1-23, wherein the compound is selected from

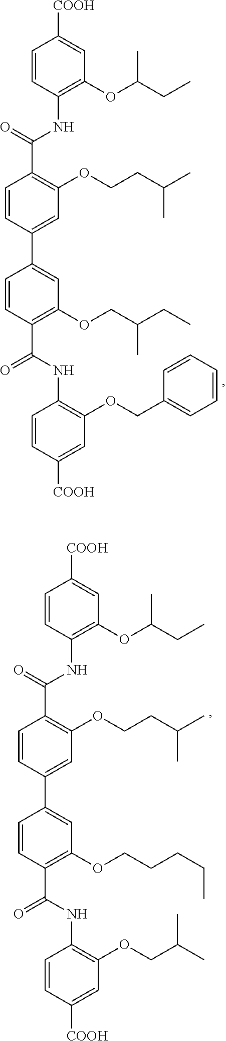 [SMILES: CCC(C)COc1cc(-c2ccc(C(=O)Nc3ccc(C(=O)O)cc3OC(C)CC)c(OCCC(C)C)c2)ccc1C(=O)Nc1ccc(C(=O)O)cc1OCc1ccccc1 ; CCCCCOc1cc(-c2ccc(C(=O)Nc3ccc(C(=O)O)cc3OC(C)CC)c(OCCC(C)C)c2)ccc1C(=O)Nc1ccc(C(=O)O)cc1OCC(C)C]

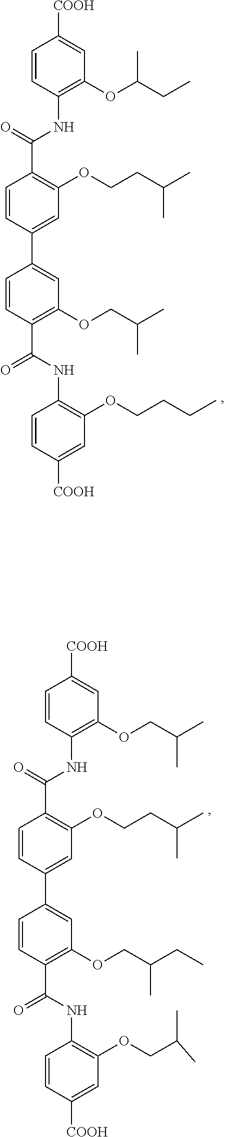 [SMILES: CCCCOc1cc(C(=O)O)ccc1NC(=O)c1ccc(-c2ccc(C(=O)Nc3ccc(C(=O)O)cc3OC(C)CC)c(OCCC(C)C)c2)cc1OCC(C)C ; CCC(C)COc1cc(-c2ccc(C(=O)Nc3ccc(C(=O)O)cc3OCC(C)C)c(OCCC(C)C)c2)ccc1C(=O)Nc1ccc(C(=O)O)cc1OCC(C)C]

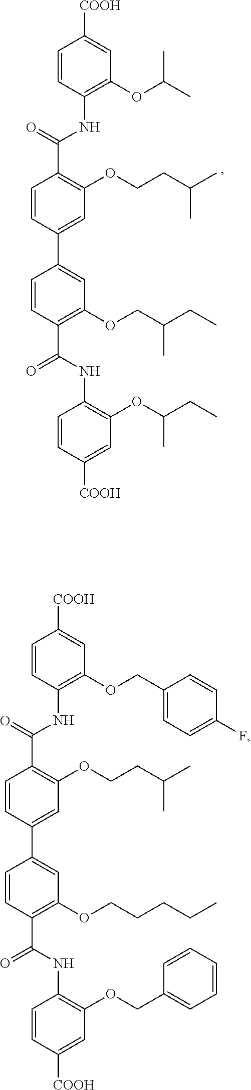 [SMILES: CCC(C)COc1cc(-c2ccc(C(=O)Nc3ccc(C(=O)O)cc3OC(C)C)c(OCCC(C)C)c2)ccc1C(=O)Nc1ccc(C(=O)O)cc1OC(C)CC ; CCCCCOc1cc(-c2ccc(C(=O)Nc3ccc(C(=O)O)cc3OCc3ccc(F)cc3)c(OCCC(C)C)c2)ccc1C(=O)Nc1ccc(C(=O)O)cc1OCc1ccccc1]

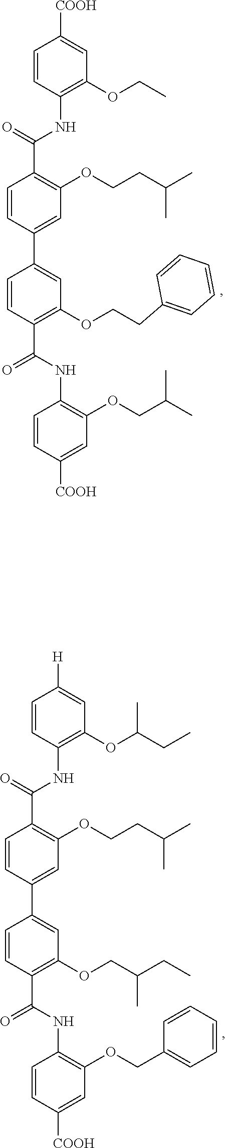 [SMILES: CCOc1cc(C(=O)O)ccc1NC(=O)c1ccc(-c2ccc(C(=O)Nc3ccc(C(=O)O)cc3OCC(C)C)c(OCCc3ccccc3)c2)cc1OCCC(C)C ; CCC(C)COc1cc(-c2ccc(C(=O)Nc3ccccc3OC(C)CC)c(OCCC(C)C)c2)ccc1C(=O)Nc1ccc(C(=O)O)cc1OCc1ccccc1]

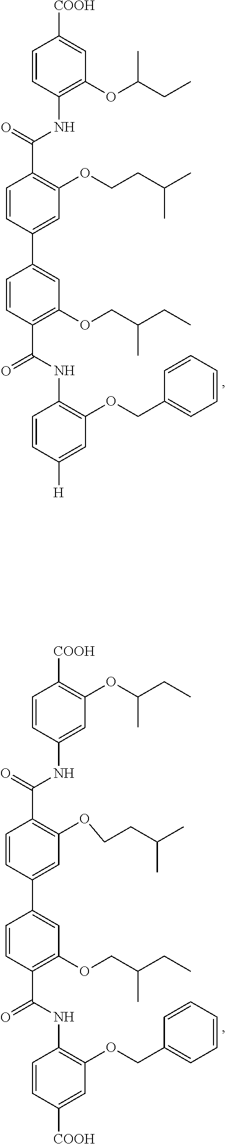 [SMILES: CCC(C)COc1cc(-c2ccc(C(=O)Nc3ccc(C(=O)O)cc3OC(C)CC)c(OCCC(C)C)c2)ccc1C(=O)Nc1ccccc1OCc1ccccc1 ; CCC(C)COc1cc(-c2ccc(C(=O)Nc3ccc(C(=O)O)c(OC(C)CC)c3)c(OCCC(C)C)c2)ccc1C(=O)Nc1ccc(C(=O)O)cc1OCc1ccccc1]

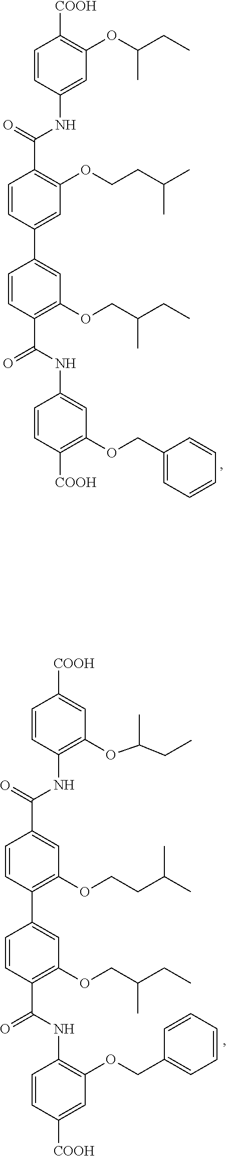 [SMILES: CCC(C)COc1cc(-c2ccc(C(=O)Nc3ccc(C(=O)O)c(OC(C)CC)c3)c(OCCC(C)C)c2)ccc1C(=O)Nc1ccc(C(=O)O)c(OCc2ccccc2)c1 ; CCC(C)COc1cc(-c2ccc(C(=O)Nc3ccc(C(=O)O)cc3OC(C)CC)cc2OCCC(C)C)ccc1C(=O)Nc1ccc(C(=O)O)cc1OCc1ccccc1]

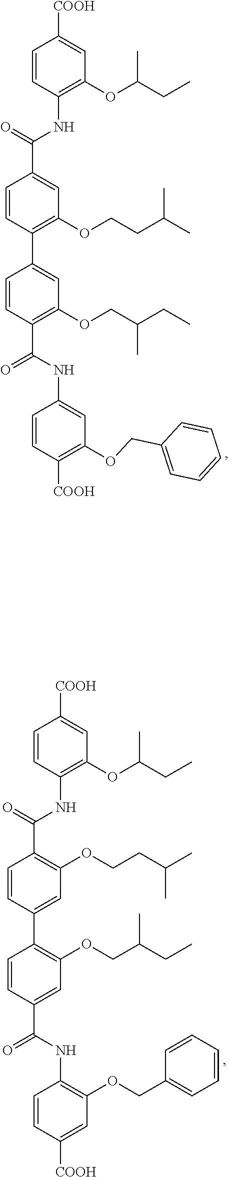 [SMILES: CCC(C)COc1cc(-c2ccc(C(=O)Nc3ccc(C(=O)O)cc3OC(C)CC)cc2OCCC(C)C)ccc1C(=O)Nc1ccc(C(=O)O)c(OCc2ccccc2)c1 ; CCC(C)COc1cc(C(=O)Nc2ccc(C(=O)O)cc2OCc2ccccc2)ccc1-c1ccc(C(=O)Nc2ccc(C(=O)O)cc2OC(C)CC)c(OCCC(C)C)c1]

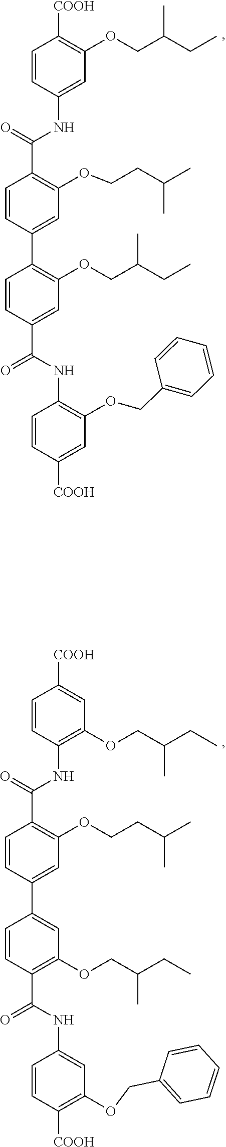 [SMILES: CCC(C)COc1cc(NC(=O)c2ccc(-c3ccc(C(=O)Nc4ccc(C(=O)O)cc4OCc4ccccc4)cc3OCC(C)CC)cc2OCCC(C)C)ccc1C(=O)O ; CCC(C)COc1cc(C(=O)O)ccc1NC(=O)c1ccc(-c2ccc(C(=O)Nc3ccc(C(=O)O)c(OCc4ccccc4)c3)c(OCC(C)CC)c2)cc1OCCC(C)C]

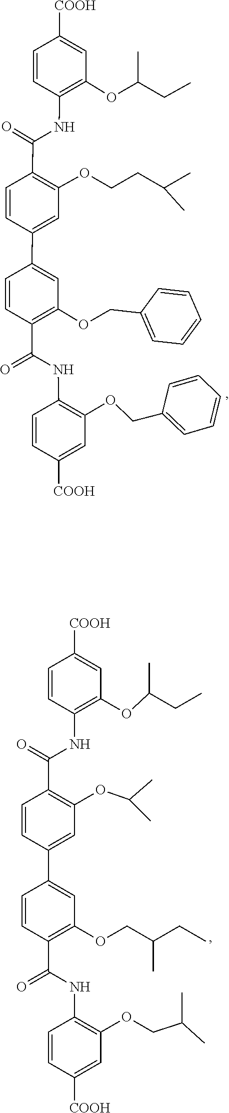 [SMILES: CCC(C)Oc1cc(C(=O)O)ccc1NC(=O)c1ccc(-c2ccc(C(=O)Nc3ccc(C(=O)O)cc3OCc3ccccc3)c(OCc3ccccc3)c2)cc1OCCC(C)C ; CCC(C)COc1cc(-c2ccc(C(=O)Nc3ccc(C(=O)O)cc3OC(C)CC)c(OC(C)C)c2)ccc1C(=O)Nc1ccc(C(=O)O)cc1OCC(C)C]

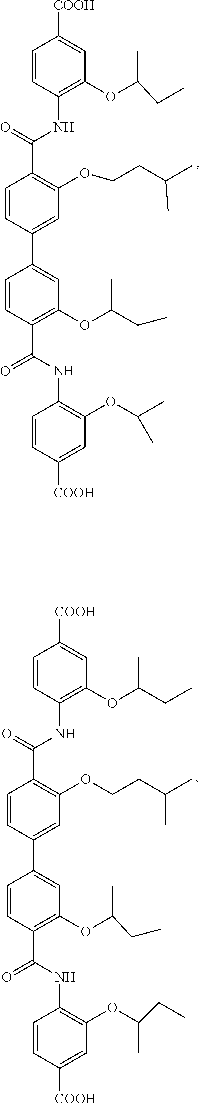 [SMILES: CCC(C)Oc1cc(C(=O)O)ccc1NC(=O)c1ccc(-c2ccc(C(=O)Nc3ccc(C(=O)O)cc3OC(C)C)c(OC(C)CC)c2)cc1OCCC(C)C ; CCC(C)Oc1cc(C(=O)O)ccc1NC(=O)c1ccc(-c2ccc(C(=O)Nc3ccc(C(=O)O)cc3OC(C)CC)c(OC(C)CC)c2)cc1OCCC(C)C]

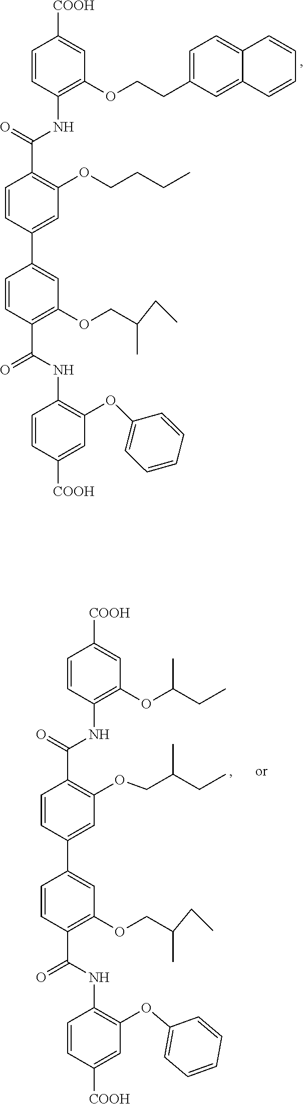 [SMILES: CCCCOc1cc(-c2ccc(C(=O)Nc3ccc(C(=O)O)cc3Oc3ccccc3)c(OCC(C)CC)c2)ccc1C(=O)Nc1ccc(C(=O)O)cc1OCCc1ccc2ccccc2c1 ; CCC(C)COc1cc(-c2ccc(C(=O)Nc3ccc(C(=O)O)cc3OC(C)CC)c(OCC(C)CC)c2)ccc1C(=O)Nc1ccc(C(=O)O)cc1Oc1ccccc1]

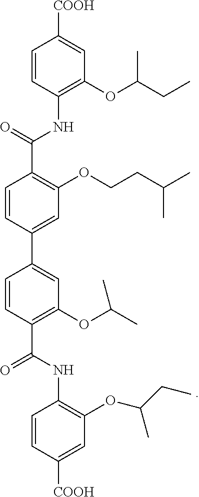 [SMILES: CCC(C)Oc1cc(C(=O)O)ccc1NC(=O)c1ccc(-c2ccc(C(=O)Nc3ccc(C(=O)O)cc3OC(C)CC)c(OC(C)C)c2)cc1OCCC(C)C]

Aspect 25. The compound of any one of aspects 1-24, wherein the compound binds to at least one anti-apoptotic protein.

Aspect 26. The compound of aspect 25, wherein the at least one anti-apoptotic protein comprises Bcl-xL, Bcl-2, Mcl-1, or any combination thereof.

Aspect 27. The compound of aspect 26, wherein the compound has an IC₅₀ for Bcl-xL of less than about 10 μM.

Aspect 28. The compound of aspect 26, wherein the compound has an IC₅₀ for Bcl-xL of less than about 1 μM.

Aspect 29. The compound of aspect 26, wherein the compound has an IC₅₀ for Bcl-2 of less than about 1 μM.

Aspect 30. The compound of aspect 26, wherein the compound has an IC₅₀ for Bcl-2 of less than about 0.1 μM.

Aspect 31. The compound of aspect 26, wherein the compound has an IC₅₀ for Mcl-1 of less than about 2 μM.

Aspect 32. The compound of aspect 26, wherein the compound has an IC₅₀ for Mcl-1 of less than about 0.2 μM.

Aspect 33. The compound of any one of aspects 25-32, wherein the compound inhibits two or more anti-apoptotic proteins.

Aspect 34. The compound of any one of aspects 25-32, wherein the compound is selective for a single anti-apoptotic protein.

Aspect 35. A pharmaceutical composition comprising the compound of Formula I or pharmaceutically acceptable salt thereof of any one of aspects 1-34.

Aspect 36. The pharmaceutical composition of aspect 35, further comprising at least one pharmaceutically acceptable excipient.

Aspect 37. The pharmaceutical composition of aspect 36, wherein the at least one pharmaceutically acceptable excipient comprises an α-cyclodextrin, a β-cyclodextrin, a γ-cyclodextrin, liposomes, a surfactant, glycerol, a polyethylene glycol, a preservative, water, ethanol, a vegetable oil, saline solution, glucose solution, a phosphate buffer, mannitol, normal serum albumin, sodium chloride solution, Ringer's dextrose, lactated Ringer's, a fixed oil, a chelating agent, or any combination thereof.

Aspect 38. A method for treating cancer in a subject, the method comprising administering a therapeutically effective amount of the compound or pharmaceutically acceptable salt of any one of aspects 1-34 or the pharmaceutical composition of any one of aspects 35-37 to the subject.

Aspect 39. The method of aspect 38, wherein the cancer comprises prostate cancer, follicular lymphoma, chronic lymphocytic leukemia, acute lymphocytic leukemia, diffuse large B cell lymphoma, multiple myeloma, acute myeloid leukemia, breast cancer, small cell lung cancer, non-small cell lung cancer, ovarian cancer, neuroblastoma, bladder cancer, colorectal cancer, head and neck cancer, or any combination thereof.

Aspect 40. The method of aspect 39, wherein the cancer is prostate cancer.

Aspect 41. The method of any one of aspects 38-40, wherein the cancer comprises a tumor in the subject and wherein the tumor after treatment has a volume of from at least 5% to at least 50% less than the volume of the tumor before treatment.

Aspect 42. The method of any one of aspects 38-40, wherein the cancer comprises a tumor in the subject and wherein the tumor after treatment has a volume of at least about 50% less than an equivalent untreated tumor.

Aspect 43. The method of any one of aspects 38-42, wherein the subject is a mammal.

Aspect 44. The method of aspect 43, wherein the mammal is a human, horse, cattle, sheep, goat, swine, dog, cat, guinea pig, rat, mouse, or rabbit.

Aspect 45. The method of any one of aspects 38-44, wherein the compound or pharmaceutical composition is administered orally or intravenously.

Aspect 46. The method of any one of aspects 38-45, further comprising administering to the subject at least one additional anticancer agent.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: α-Helix Mimetics for Protein Interactions

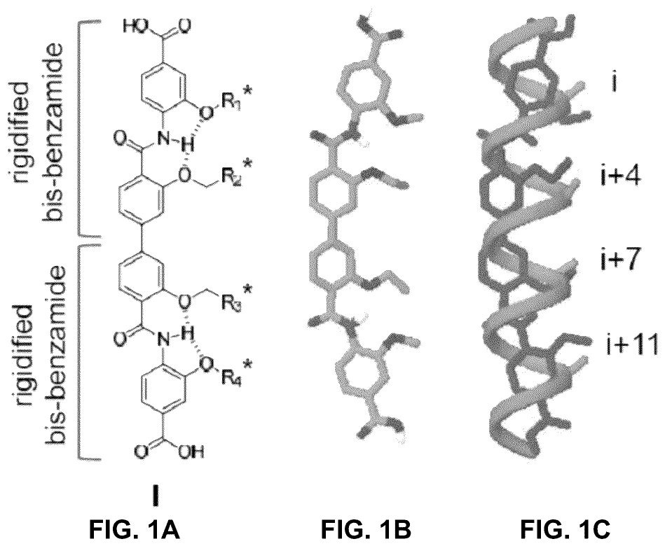

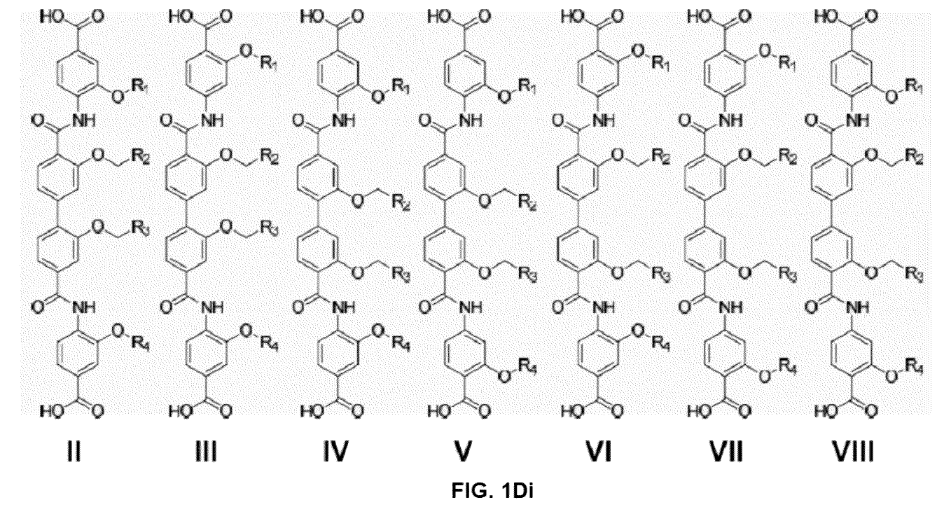

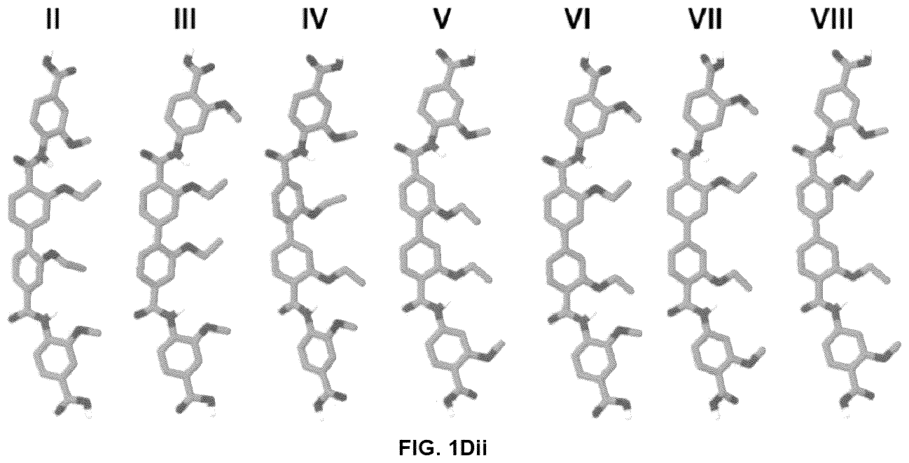

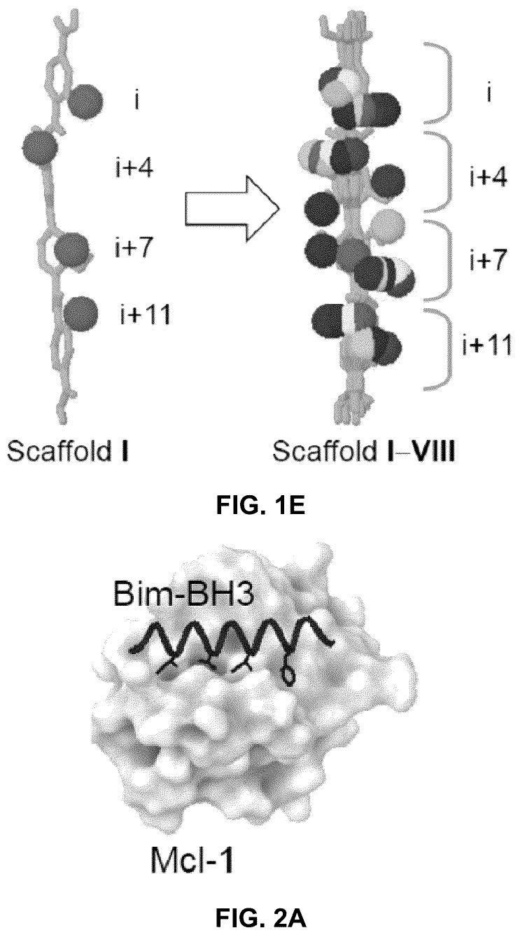

While α-helix mimetics have been typically designed to target short α-helices of one or two helical turns, α-helices found at PPI interfaces often extend to three or longer helical turns. Thus, a longer scaffold mimicking long helical segments may acquire higher binding affinity and selectivity for target proteins. To mimic extended α-helical surfaces, an N,N′-diphenyl-4,4′-biphenyldicarboxamide scaffold I that presents four substituents corresponding to the side chains at the i, +4, +7, and i+11 positions of three-turn helices (FIG. 1A) was designed. The scaffold I comprises of two bis-benzamides connected via a C—C bond. The bis-benzamide is rigidified by intramolecular five- and six-membered hydrogen bonds and aligns two adjacent alkoxy substituents (R₁/R₂ and R₃/R₄) on the same side. To demonstrate its α-helix mimicry, a Monte Carlo conformational search of the scaffold I was carried out by using MacroModel (version 9.4, Schrodinger, NY). The resulting 100 conformations were clustered by using clustering of conformer's script in Maestro (version 9.4, Schrodinger, NY). A representative conformation of the most populated cluster showed that four substituents are well overlaid over the corresponding side chains of an α-helix (FIGS. 1B-1C).

Despite the outstanding structural mimicry of α-helix, the scaffold I places its substituents only in a limited spatial arrangement (FIG. 1E) that may be insufficient to account for slightly adjusted helical structures when interacting with protein partners. A collection of scaffolds, rather than one, would represent more diverse conformations. Therefore, scaffolds II-VIII, regioisomers of the scaffold I, that share the biphenyl backbone but have combinations of regioisomeric 2- and 3-alkoxybenzene ring (FIGS. 1Di-1Dii) were designed. Whereas the conformational search showed that scaffolds II-VIII still place four substituents on the same side as the scaffold I does, maintaining α-helix mimicry, an overlay of the representative conformations of the scaffolds I-VIII demonstrated that scaffolds I-VIII present diverse side chain arrangement which may appear in various natural α-helical conformations. This diversity is not readily accommodated by existing α-helix-mimicking scaffolds (FIG. 1E).

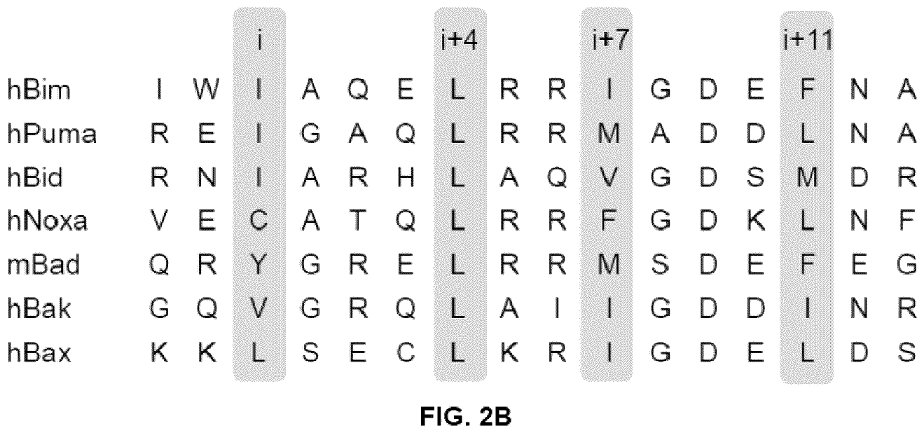

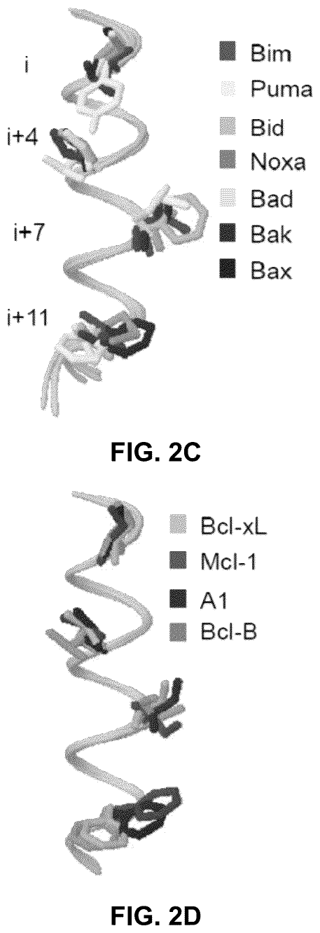

To validate and demonstrate this strategy, an attempt was made to modulate the interaction between Bcl-2 family proteins. These proteins are critical in the regulation of the intrinsic apoptosis, and comprise of anti- and pro-apoptotic proteins. Anti-apoptotic proteins (e.g., Bcl-2, Bcl-xL, Bcl-w, Mcl-1, and A1) block apoptosis by making complexes with pro-apoptotic members. Pro-apoptotic proteins are further classified to two subgroups: effectors (e.g., Bax and Bak) directly facilitating the mitochondrial outer membrane permeabilization (MOMP) and BH3-only proteins (e.g., Bim, Bid, Bik, Puma, Noxa, and Bmf) regulating the other two classes. Interaction between anti- and pro-apoptotic Bcl-2 family proteins are mediated by the α-helical BH3 domain of pro-apoptotic members that binds to a hydrophobic pocket formed by BH1-3 domains in anti-apoptotic ones (FIG. 2A). Despite the structural similarity, the BH3 domains differ in the amino acid sequence and the spatial arrangement of the side chains, bringing in differential binding affinities (FIGS. 2B-2D). A balance between anti- and pro-apoptotic proteins determines whether cells undergo apoptosis in response to apoptotic stimuli. Anti-apoptotic proteins are frequently overexpressed in cancer cells and contribute to tumorigenesis and drug resistance, making them attractive therapeutic targets. Consequently, venetoclax (ABT-199) became the first BH3 mimetic approved by the FDA for the treatment of chronic lymphocytic leukemia.

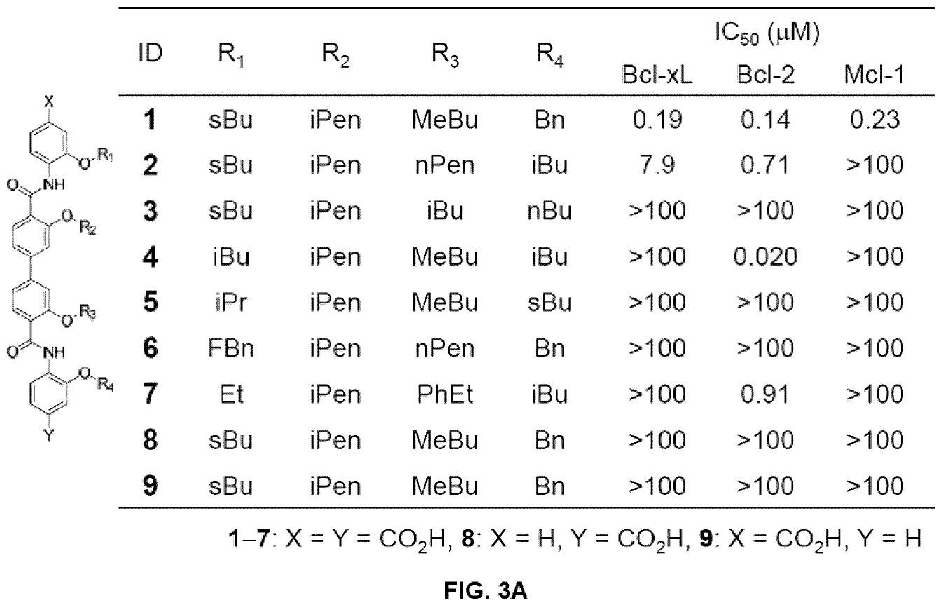

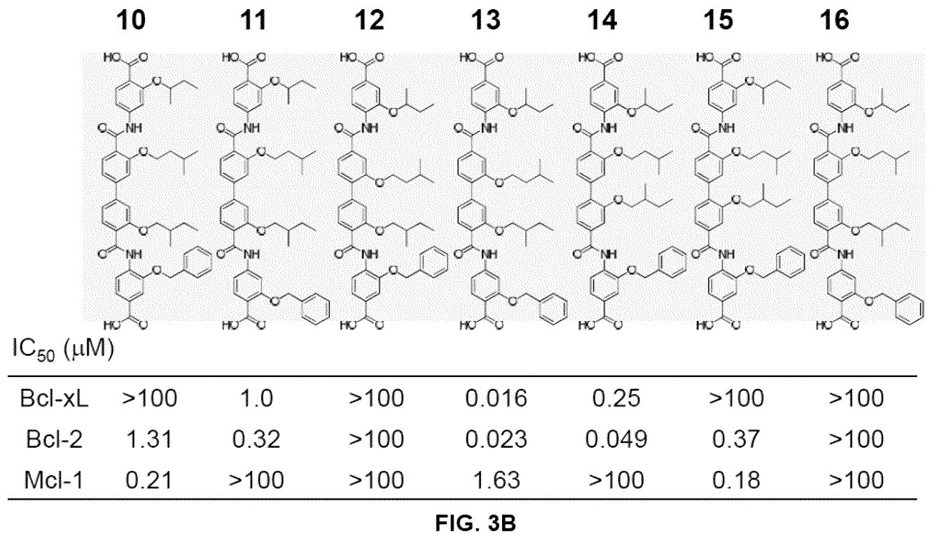

Our initial efforts focused on recapitulating the distinct binding patterns of pro-apoptotic proteins. Compounds 1-7 were designed based on the scaffold I to present four substituents corresponding to the key hydrophobic side chains of the BH3 peptides of Bim, Puma, Bid, Bax, Bak, Bad, and Noxa, respectively (FIGS. 2B and 3A). The biphenyl compounds were convergently synthesized by Suzuki cross-coupling reactions between two bis-benzamides that were prepared with 4-iodosalicylic acid and 3-hydroxy-4-nitrobenzoic acid (Scheme 1). Due to synthetic difficulties, 4-fluorobenzyl, n-pentyl, and ethyl groups were introduced to represent the side chain groups of Tyr, Met, and Cys, respectively.

Their binding affinities on Bcl-xL, Bcl-2, and Mcl-1 were determined by fluorescence polarization (FP) assays (FIG. 3A). Bim, Puma, Bid, Bax, and Bak are known to bind to the three anti-apoptotic proteins, but their BH3 mimetics 1-5 did not always reflect the binding profiles of the parent proteins. Bim BH3 mimetic 1 has high binding affinity for all three anti-apoptotic proteins with the IC₅₀ values ranging from 0.14 to 0.23 μM, displaying the same binding profile as the parent protein, Bim. However, Puma BH3 mimetic 2 showed a low affinity for Bcl-xL and Mcl-1, while keeping high affinity for Bcl-2. Bid BH3 mimetic 3 showed no inhibition against the three proteins. On the other hand, Bax BH3 mimetic 4 was very selective for inhibiting Bcl-2 over Bcl-xL and Mcl-1. Bak BH3 mimetic 5 has negligible affinity towards any of the three proteins. Whereas Bad appears to interact with both Bcl-xL and Bcl-2, its BH3 mimetic 6 did not bind to the three proteins. Noxa selectively binds to Mcl-1, however its BH3 mimetic 7 showed affinity only for Bcl-2. The role of carboxylic acids at both ends of compound 1 was also studied. Its removal (8 and 9) led to complete loss in binding affinities, highlighting the importance of both carboxylic acids. Since Bim BH3 mimetic 1 exhibited strong binding affinity for all three anti-apoptotic proteins, 1 was selected for further study.

The four substituents selected from the Bim BH3 mimetic 1 (i.e., sec-butyl, isopentyl, 2-methylbutyl, and benzyl groups) were placed on the scaffolds II-VIII to make subtle changes in their spatial arrangement as shown in FIGS. 1Di-1E. Such small topological variations are expected to differentiate the close-looking binding pockets organized in the three anti-apoptotic Bcl-2 family proteins. Compounds 10-16 (FIG. 3B) were synthesized in an analogous manner as 1 with the modified monomers, 3-hydroxy-4-iodobenzoic acid, 2-hydroxy-4-nitrobenzoic acid, 4-iodosalicylic acid, and 3-hydroxy-4-nitrobenzoic acid (Schemes 2A-2B).

The hydrophobic surfaces slightly differently constituted by the scaffolds I-VIII showed dramatic effects on binding patterns (FIG. 3B). Changing the position of the sec-butyl group from the 3-position (1) to 2-position (10) lost affinity for Bcl-xL while maintaining affinity for Bcl-2 and Mcl-1. Furthermore, moving the benzyl group of 10 from the 3- to 2-position also altered the binding pattern as the resulting compound 11 showed selectivity for Bcl-xL and Bcl-2. When the position of the isopentyl group of 1 was changed, the resulting compound 12 significantly lost affinity for all three proteins. However, when the benzyl group of 12 was changed from the 3- to 2-position, the resulting compound 13 substantially increased its affinity to Bcl-xL and Bcl-2 compared to Mcl-1. Compound 14 containing the 2-methylbutyl group at the 3-position displayed strong affinity to Bcl-2, but not to Mcl-1, showing a similar binding pattern to that of 11. On the other hand, changing the position of the sec-butyl group of 14 altered the target selectivity, making 5 selective to Bcl-2 and Mcl-1, but not to Bcl-xL. When only the benzyl group of 1 was moved from 3- to 2-position, the resulting compound 16 surprisingly lost affinity to all three proteins.

It is remarkable that while compounds 10-16 have the identical four substituents, they showed distinct binding profiles. This may imply that the scaffolds I-VIII constitute subtly altered spatial arrangements of the four same substituents, resulting in differentiation of the similarly organized binding pockets in the three anti-apoptotic proteins and achieving various target selectivity with the same set of the substituents.

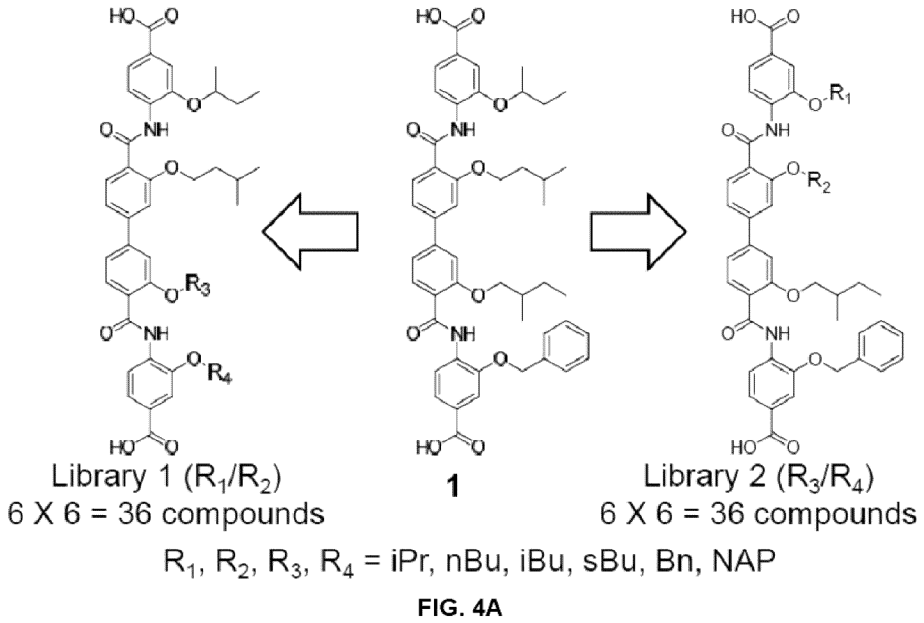

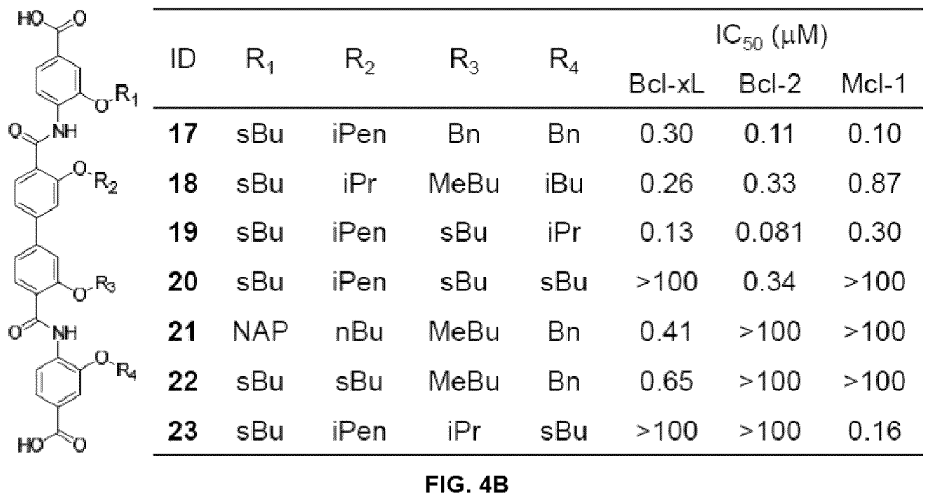

In addition to examining the tunability of the biphenyl scaffolds by varying the positions of the fixed set of the four substituents, the substituents of the scaffold I-based Bim BH3 mimetic 1 were varied to carry out a topological survey for facilitating high binding affinity and target selectivity. 36-Membered library 1 was constructed by varying R₁ and R₂ while R₃ and R₄ were fixed as 2-methylbutyl and benzyl groups, respectively. Similarly, 36-membered library 2 was prepared by varying R₃ and R₄ while R₁ and R₂ were held constant as sec-butyl and isopentyl groups, respectively (FIG. 4A). The libraries 1 and 2 were quickly screened at a single concentration to identify potent compounds, which were followed by dose-dependent assays to determine binding affinity (FIG. 4B). Among 72 compounds, BH3 mimetics 17-19 displayed strong binding affinity for all of anti-apoptotic proteins, Bcl-xL, Bcl-2, and Mcl-1 with the IC₅₀ values ranging from 0.081 μM to 0.87 μM.

In addition to these pan-active inhibitors, highly selective inhibitors to each anti-apoptotic Bcl-2 family protein were also identified. Compound 20 was found to be a Bcl-2 selective inhibitor (IC₅₀=0.34 μM) with negligible binding to Bcl-xL and Mcl-1. On the other hand, compounds 21 and 22 exhibited selective affinity only to Bcl-xL with the IC₅₀ values of 0.41 μM and 0.65 μM, respectively. Remarkably, compound 23 selectively inhibited only Mcl-1 with the IC₅₀ value of 0.16 μM, demonstrating the utility and versatility of the biphenyl scaffold.

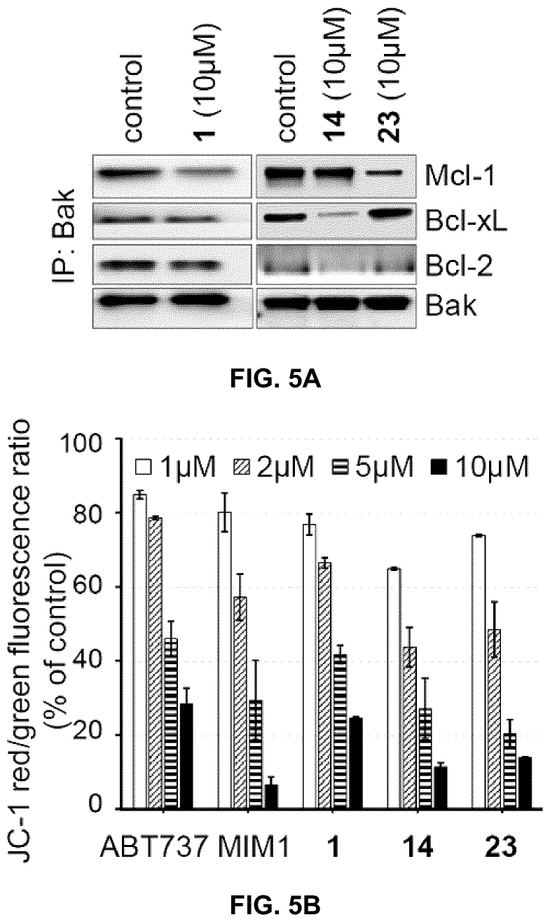

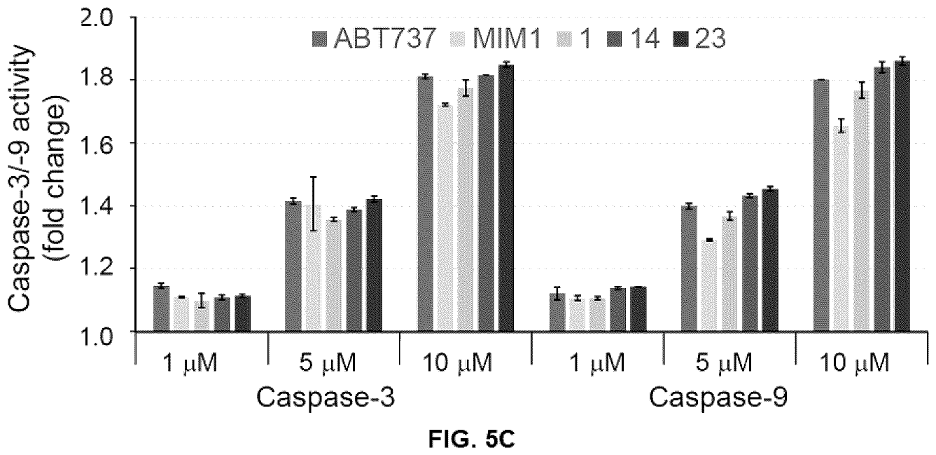

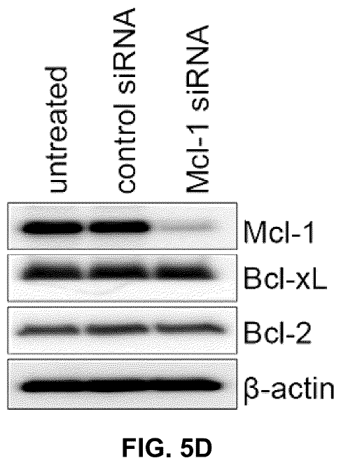

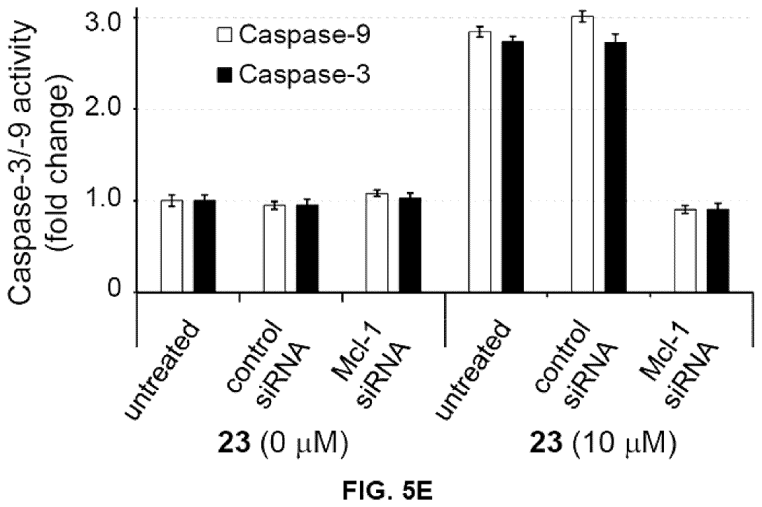

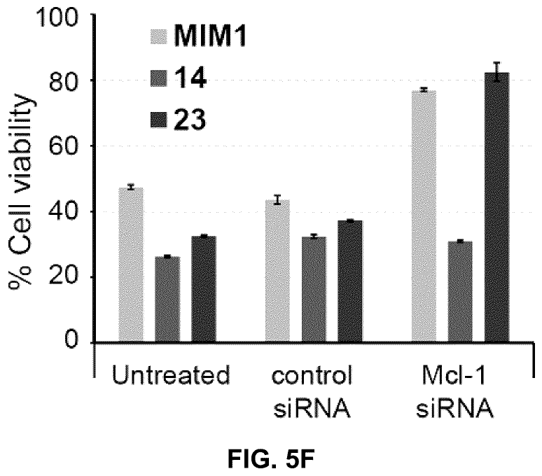

BH3 mimetics 1, 14, and 23 were selected as pan-active, Bcl-2 and Bcl-xL-selective, and Mcl-1-specific inhibitors, respectively, and they were further examined in cells to investigate if their binding profiles correlate to their biochemical activities. Co-immunoprecipitation (Co-IP) experiments using PC3 cells showed that 1 disrupted the interaction of Bak with Bcl-xL, Bcl-2, and Mcl-1, acting as a pan-inhibitor (FIG. 5A). Compound 14 disrupted the interaction of both Bcl-xL and Bcl-2, but not Mcl-1, with Bak. Compound 23 selectively inhibited Mcl-1 over Bcl-xL and Bcl-2. Notably, these results are consistent with the binding profiles already observed by the FP assay.

These compounds were examined for their capabilities of inducing apoptosis. When effectors are released from anti-apoptotic proteins, they form pores on the outer mitochondrial membrane, leading to mitochondrial outer membrane permeabilization (MOMP) and cytochrome c release which promote caspase activation. The formation of pores on the outer mitochondrial membrane and the resulting change in mitochondrial membrane potential were determined by JC-1 staining. Compounds 1, 14, and 23 significantly reduced the red to green fluorescence intensity ratio of JC-1 dye with comparable potency to that of ABT737, a dual Bcl-xL/Bcl-2 inhibitor, and MIM1, a selective Mcl-1 inhibitor, suggesting that the compounds can induce MOMP as expected (FIG. 5B). Furthermore, BH3 mimetics 1, 14, and 23 also induced caspase-9/-3 activity in a dose-dependent manner, demonstrating their on-target activity (FIG. 5C). The anti-proliferative activity was also evaluated using MTT assay in prostate cancer cells. Compounds 1, 14, and 23 suppressed cell growth in LNCaP, DU145, and PC3 cells with the IC₅₀ values ranging from 2.87 μM to 9.67 μM, which are comparable to ABT737 and MIM1.

Compound 23 was found to be highly selective to Mcl-1 evidenced by the FP assays and Co-IP experiments (vide supra). Its specificity to Mcl-1 on prostate cancer cells with Mcl-1 silenced by siRNA (FIG. 5D) was further investigated. Compound 23 induced up to 3-fold increase in caspase-3/-9 activity in untransfected and control-transfected PC3 cells. Knockdown of Mcl-1, however, decreased the M161-induced caspase activity to the basal level (FIG. 5E). In MTT assay, 23 inhibited cell growth by 64% and 62% in untransfected and control-transfected PC3 cells, respectively. However, knockdown of Mcl-1 abolished the 23-induced growth inhibition, showing only 18% inhibition. Similar results were observed with the Mcl-1-selective inhibitor MIM1, validating the Mcl-1 specificity of BH3 inhibitor 23. In contrast, the inhibitory activity of 14, identified as a dual Bcl-xL/Bcl-2 inhibitor (vide supra), was unaffected by the expression level of Mcl-1 (FIG. 5F). Collectively, these data confirm that compound 23 selectively targets Mcl-1.

In summary, a novel scaffold based on the N,N′-diphenyl-4,4′-biphenyldicarboxamide structure was designed for reproducing longer α-helical surfaces by accommodating four substituents corresponding to the i, i+4, i+7, and i+11 positions in the α-helix. The larger helical surface replicated by the scaffold not only promotes stronger interaction with target proteins, but also provide higher target selectivity. In addition, this biphenyl scaffold is proven to be unique and versatile as its regioisomers can make subtle change in the positions of the four substituents, facilitating fine custom-fit to binding interfaces and consequently discriminating a target protein among others with closely resembling binding interfaces. To demonstrate its potential, it was evaluated on Bcl-2 family proteins and identified pan-active and selective inhibitors to each member of anti-apoptotic Bcl-2 proteins. These results validate the utility of the new biphenyl scaffold in replicating longer α-helical surfaces and in turn disrupting protein-protein interaction with high efficacy and selectivity. This strategy is especially useful for selectively targeting a specific protein among many exhibiting similar binding interfaces for protein-protein interactions.

### Example 2: Synthetic Schemes and Procedures

Exemplary schemes for producing the disclosed compounds and synthetic intermediates are shown in Schemes 1-2B below.

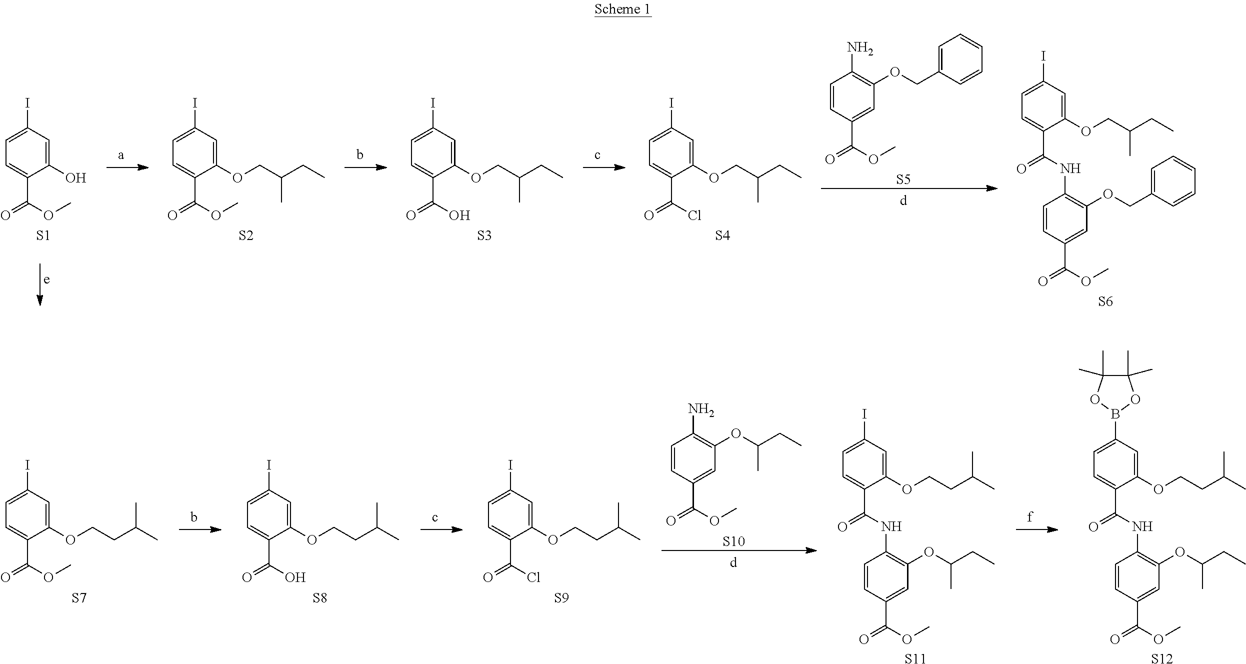

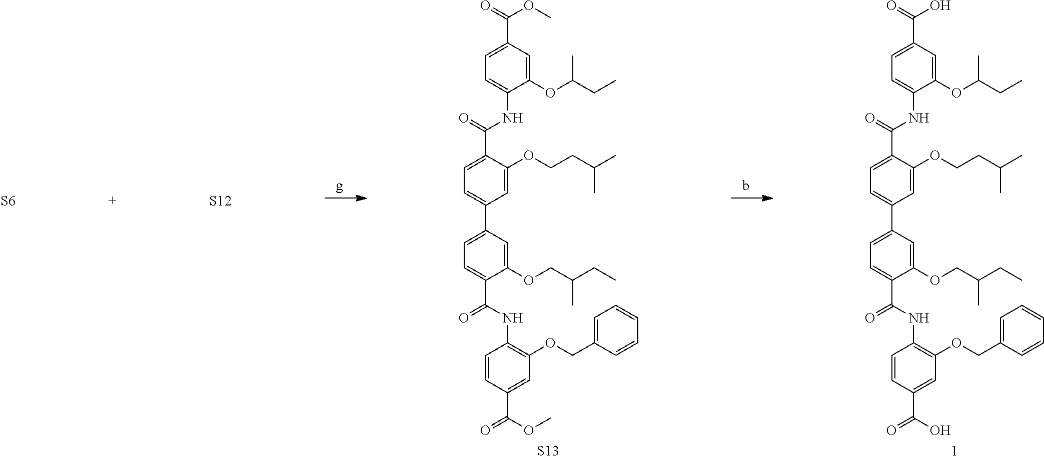

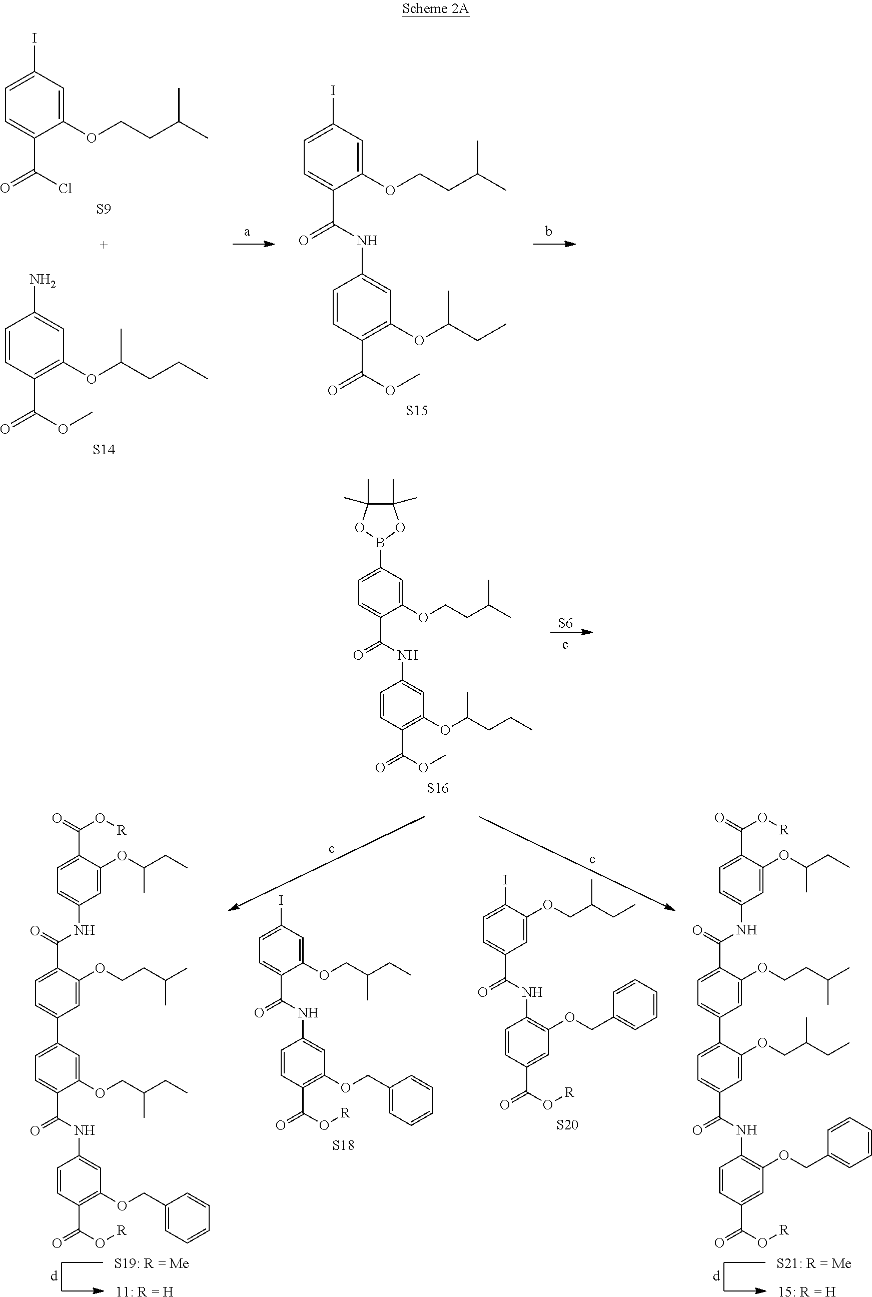

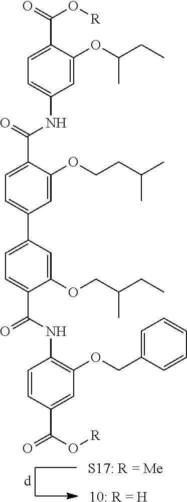

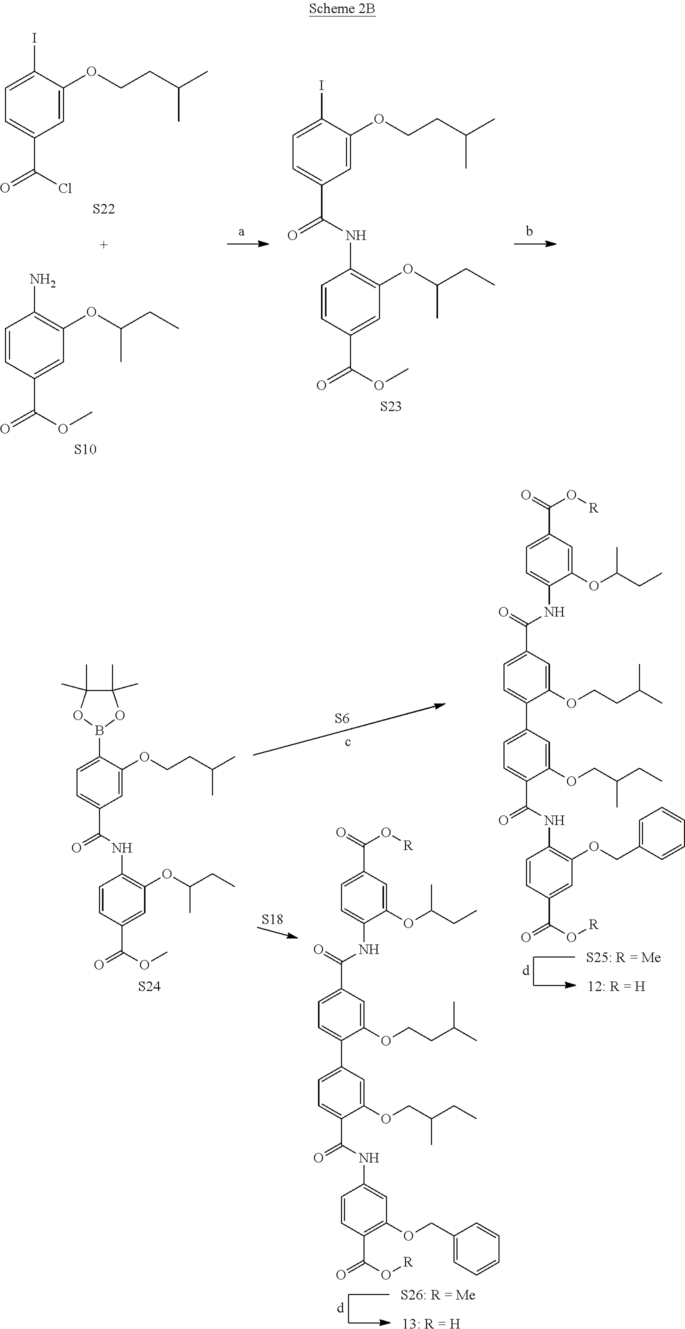

Scheme 1. Synthesis of compound 1. Reagents and conditions: (a) 1-chloro-2-methylbutane, K₂CO₃, DMF, 80° C., 12 h; (b) NaOH, H₂O, MeOH/THF, rt, 12 h; (c) (COCl)₂, cat. DMF, CH₂Cl₂, rt, 2 h; (d) DIEA, CH₂Cl₂, rt, 12 h; (e) 1-rromo-3-methylbutane, K₂CO₃, DMF, 80° C., 12 h; (f) PdCl₂-dppf, bis(pinacolato)diboron, NaOAc, DMSO, 80° C., 12 h; (g) PdCl₂-dppf, K₂CO₃, DMF, 80° C., 12 h.

Schemes 2A-2B. Synthesis of compounds 10-16. Reagents and conditions: (a) DIEA, CH₂Cl₂, rt, 12 h; (b) PdCl₂-dppf, bis(pinacolato)diboron, NaOAc, DMSO, 80° C., 12 h; (c) PdCl₂-dppf, K₂CO₃, DMF, 80° C., 12 h; (d) NaOH, H₂O, MeOH/THF, rt, 12 h.

General Procedure A: Amide Bond Formation with Acid Chloride. Oxalyl chloride (2.0 mmol, 4.0 equiv) and two drops of anhydrous DMF were slowly added to a suspension of a carboxylic acid (1.0 mmol, 2.0 equiv) in DCM (30 mL). The reaction mixture was stirred at room temperature for 2 h, and the solvent and excess oxalyl chloride were removed under reduced pressure. The resulting acid chloride was then dissolved in DCM (10 mL) and slowly added to a solution of an amine (0.50 mmol, 1.0 equiv) and DIEA (1.5 mmol, 3.0 equiv) in DCM (80 mL). The reaction mixture was stirred at room temperature for 24 h and concentrated under reduced pressure. The resulting residue was then diluted with EtOAc (100 mL) and aqueous HCl solution (1 N, 100 mL). The organic layer was separated, washed with saturated NaHCO₃ (50 mL) and brine (50 mL), and concentrated under reduced pressure. The resulting solid was washed with EtOAc (3×20 mL) and dried in vacuo to give the desired product.

General Procedure B: Amide Bond Formation by PyBrOP. An amine (0.25 mmol, 1.0 equiv) was added to a solution of a carboxylic acid (0.50 mmol, 2.0 equiv), PyBrOP (0.50 mmol, 2.0 equiv), and DIEA (1.0 mmol, 4.0 equiv) in DMF (10 mL), and the reaction mixture was stirred at room temperature for 24 h. The reaction mixture was then diluted with EtOAc (100 mL) and aqueous HCl solution (1 N, 100 mL). The organic layer was separated, washed with saturated NaHCO₃ solution (50 mL) and brine (50 mL), and concentrated under reduced pressure. The resulting solid was washed with EtOAc (3×10 mL) and dried in vacuo to give the desired product.

General procedure C: O-Alkylation. To the mixture of methyl 4-iodosalicylate (S1) (18.0 mmol, 1.0 equiv) and K2CO3 (36.2 mmol, 2.0 equiv) in DMF (150 mL) was added an alkyl halide (54.0 mmol, 3.0 equiv). The reaction mixture was heated to 90° C. for 12 h. After cooling to room temperature, the reaction mixture was then diluted with EtOAc (600 mL) and brine (600 mL). The organic layer was separated, washed with brine (2×300 mL), concentrated under reduced pressure, and used in the next reaction without further purification.

General procedure D: Hydrolysis of Esters with NaOH. To the solution of an O-alkylated methyl benzoate (14.4 mmol, 1.0 equiv) in THF (50 mL) and MeOH (100 mL) was added aqueous NaOH solution (10%, 20 mL), and the reaction mixture was stirred at room temperature for 12 h. The reaction mixture was then concentrated to a volume of 40 mL under reduced pressure and washed with hexanes (40 mL). The resulting residues was acidified to pH 1 with aqueous HCl solution (1 N) and extracted with EtOAc (200 mL). The organic layer was washed with brine (2×100 mL), dried over Na2SO4, filtered, and concentrated under reduced pressure. The resulting solid was washed with hexane and filtered and dried over a Hirsch funnel.

General Procedure E: Borylation. Bis(diphenylphosphino)ferrocene]dichloropalladium(II) (0.02 mmol, 0.1 equiv) was added to the mixture of an aryl iodide (0.2 mmol, 1.0 equiv), bis(pinacolato)diboron (0.24 mmol, 1.2 equiv), and sodium acetate (1.2 mmol, 6.0 equiv) in DMSO. The reaction mixture was stirred at 80° C. for 12 h, it was cooled to room temperature and extracted with EtOAc (200 mL) and brine (200 mL). The organic layer was separated, washed with brine (2×150 mL), concentrated under reduced pressure, and purified by column chromatography.

General Procedure F: Suzuki Cross-Coupling Reaction. An aryl iodide (0.3 mmol, 1.0 equiv) was added to the mixture of borylated compound (0.36 mmol, 1.2 equiv), bis(diphenylphosphino)ferrocene]dichloropalladium(II) (0.03 mmol, 0.1 equiv), and K₂CO₃ (0.9 mmol, 3.0 equiv) in DMF, and the reaction mixture was stirred at 80° C. for 12 h. It was cooled to room temperature and extracted with EtOAc (200 mL) and water (200 mL). The organic layer was separated, washed with brine (2×150 mL), concentrated under reduced pressure. The product was precipitated by EtOAc/Hexane (20%).

General Procedure G: Hydrolysis of Esters with LiOH. To the solution of methyl benzoate (0.2 mmol, 1.0 equiv) in THF/MeOH (1:1, 10 mL) was added aqueous LiOH solution (1 N, 2.0 mL), and the reaction mixture was stirred at room temperature for 12 h. The reaction mixture was then concentrated under reduced pressure and diluted with water. The aqueous layer was washed with ether (2×50 mL), acidified to pH 1 with aqueous HCl solution (1 N), and extracted with EtOAc (2×50 mL). The combined organic layer was washed with brine (2×100 mL), dried over Na₂SO₄, and concentrated under reduced pressure.

### Exemplary Compounds

4-Iodo-2-(2-methylbutoxy)benzoic acid (S3). The title compound was synthesized from Methyl 4-iodo-2-(2-methylbutoxy)benzoate (S2) by following the O-Alkylation of methyl 4-iodosalicylate (S1) (General Procedure C) and hydrolysis of ester (General Procedure D). The title compound was obtained as yellow solid (5.9 g, 98% yield).

Methyl 4-amino-3-(benzyloxy)benzoate (S5). The title compound was prepared by following a literature procedure. The title compound was obtained as brown solid (4.7 g, 98% yield).

Methyl 3-(benzyloxy)-4-(4-iodo-2-(2-methylbutoxy)benzamido)benzoate (S6). The title compound was synthesized with S3 (2.0 g, 6.0 mmol) and S5 (1.0 g, 6.0 mmol) by following amide bond formation reaction (General Procedure A), and obtained as white solid (530 mg, 84% yield).

4-Iodo-2-(isopentyloxy)benzoic acid (S8). The title compound was synthesized from Methyl 4-iodo-2-(isopentyloxy)benzoate (S7) by following the O-Alkylation (General Procedure C) and hydrolysis of ester (General Procedure D). The title compound was obtained as yellow solid (5.9 g, 98% yield).

Methyl 4-amino-3-(sec-butoxy)benzoate (S10). The title compound was prepared by following a literature procedure.

Methyl 3-(sec-butoxy)-4-(4-iodo-2-(isopentyloxy)benzamido)benzoate (S11). The title compound was synthesized with S9 and S10 by following amide bond formation (General Procedure A) and purified by column chromatography. The title compound was obtained as colorless oil (950 mg, 84% yield).

Methyl 3-(sec-butoxy)-4-(2-(isopentyloxy)-4-(4,4,5,5-tetramethyl-1,3,2-dioxaborolan-2-yl)benzamido)benzoate (S12). The title compound was synthesized with S11 by following borylation reaction (General Procedure E) and obtained as light yellow oil (430 mg, 54% yield).

3-(Benzyloxy)-4-(4′-((2-(sec-butoxy)-4-carboxyphenyl)carbamoyl)-3′-(isopentyloxy)-3-(2-methylbutoxy)-[1,1′-biphenyl]-4-carboxamido)benzoic acid (1). The title compound was synthesized from S6 and S12 by following the Suzuki cross-coupling reaction (General Procedure F) and the hydrolysis reaction (General Procedure G) to obtained as a white solid (145 mg, 88% yield).

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

### REFERENCES

1. Ashkenazi, A et al, Nature Reviews Drug Discovery 2017, 16, 273.
2. Azzarito, V et al, Chemical Science 2015, 6, 2434-2443.
3. Azzarito, V et al, Nat. Chem. 2013, 5, 161.
4. Chipuk, J E et al, Molecular Cell 2010, 37, 299-310.
5. Cohen, N A et al, Chemistry & Biology 2012, 19, 1175-1186.
6. Cossu, A et al, PLOS ONE 2012, 7, e48817.
7. Davis, J M et al, Chemical Society Reviews 2007, 36, 326-334.
8. Jochim, A L et al, Molecular BioSystems 2009, 5, 924-926.
9. Ko, E et al, Journal of the American Chemical Society 2013, 135, 167-173.
10. Marimganti, S et al, Organic Letters 2009, 11, 4418-4421.
11. Mizuno, A et al, Chemistry—A European Journal 2017, 23, 3159-3168.
12. Oltersdorf, T et al., Nature 2005, 435, 677-681.
13. Pelay-Gimeno, M et al, Angew. Chem. Int. Ed. 2015, 54, 8896-8927.
14. Placzek, W J et al, Journal of Biological Chemistry 2011, 286, 39829-39835.
15. Rodriguez, J M et al, Angewandte Chemie International Edition 2007, 46, 8614-8617.
16. Rodriguez, J M et al, ChemBioChem 2009, 10, 829-833.
17. Saraogi, I et al, Angewandte Chemie International Edition 2008, 47, 9691-9694.
18. Sattler, M et al, Science 1997, 275, 983.
19. Scott, D E et al, Nat. Rev. Drug Discov. 2016, 15, 533.
20. Singh, R et al, Nature Reviews Molecular Cell Biology 2019, 20, 175-193.
21. Souers, A J et al., Nature Medicine 2013, 19, 202.
22. Tyndall, J D A et al, Chemical Reviews 2005, 105, 973-1000.
23. Walensky, L D et al, Science 2004, 305, 1466.
24. Wei, G et al, Chemical Reviews 2016, 116, 6516-6551.

What is claimed is:

1. A compound having Formula I or a pharmaceutically acceptable salt thereof 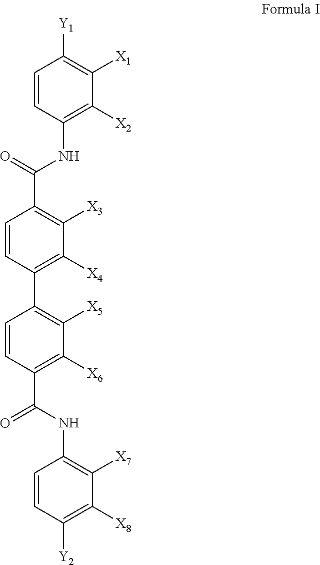
wherein each of X₁-X₈ is individually selected from H, OR₁, NR₁R₂, SR₁, or PR₁R₂;
wherein each of Y₁-Y₂ is individually selected from H, CO₂R₁, C(═O)NR₁R₂, C(═O)R₁, OR₁, NR₁R₂, SR₁, or PR₁R₂;
wherein each of R₁ and R₂ is individually selected from H, linear or branched C1-C20 alkyl, C3-C20 aryl, linear or branched C2-C20 alkenyl, linear or branched C2-C20 alkynyl, or C3-C20 arylalkyl; and
wherein when R₁, R₂, or both R₁ and R₂ are linear or branched C1-C20 alkyl, C3-C20 aryl, linear or branched C2-C20 alkenyl, linear or branched C2-C20 alkynyl, or C3-C20 arylalkyl, R₁, R₂, or both R₁ and R₂ can be optionally substituted with an ether, amine, alkoxy, ester, amide, thiol, phosphine, or any combination thereof.

2. The compound or pharmaceutically acceptable salt of claim 1, wherein at least four of X₁-X₈ are H.

3. The compound or pharmaceutically acceptable salt of claim 1, wherein at least four of X₁-X₈ are OR₁.

4. The compound or pharmaceutically acceptable salt of claim 1, wherein Y₁ and Y₂ are CO₂H.

5. The compound or pharmaceutically acceptable salt of claim 1, wherein Y₁ is H and Y₂ is CO₂H.

6. The compound or pharmaceutically acceptable salt of claim 1, wherein Y₁ is CO₂H and Y₂ is H.

7. The compound or pharmaceutically acceptable salt of claim 3, wherein each R₁ is individually selected from sec-butyl, isobutyl, isopropyl, ethyl, isopentyl, methylbutyl, n-pentyl, phenylethyl, benzyl, naphthyl, or n-butyl.

8. The compound or pharmaceutically acceptable salt of claim 1, wherein the compound is selected from

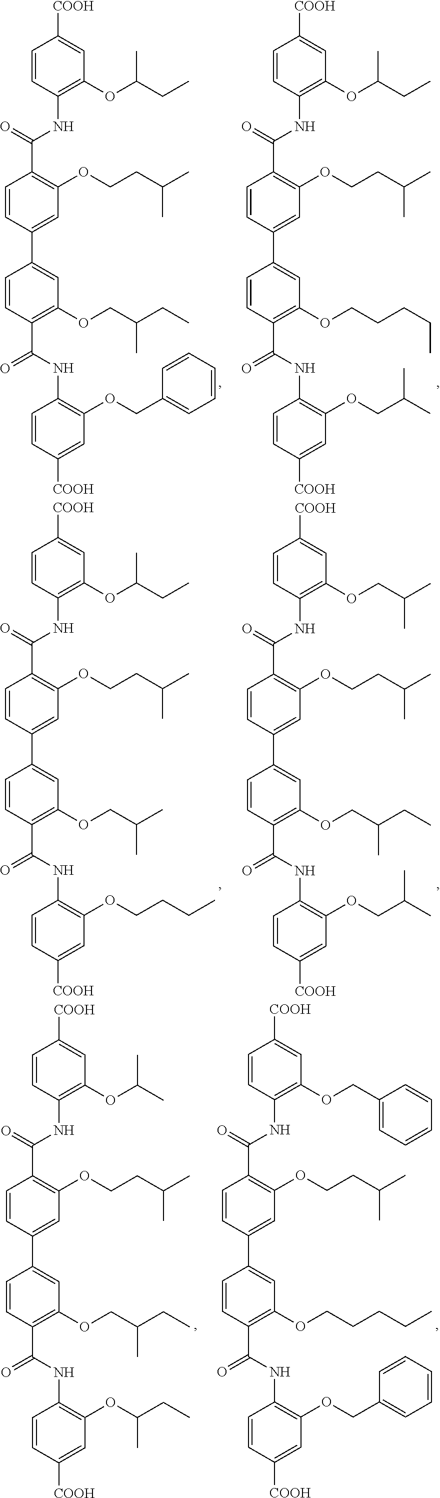

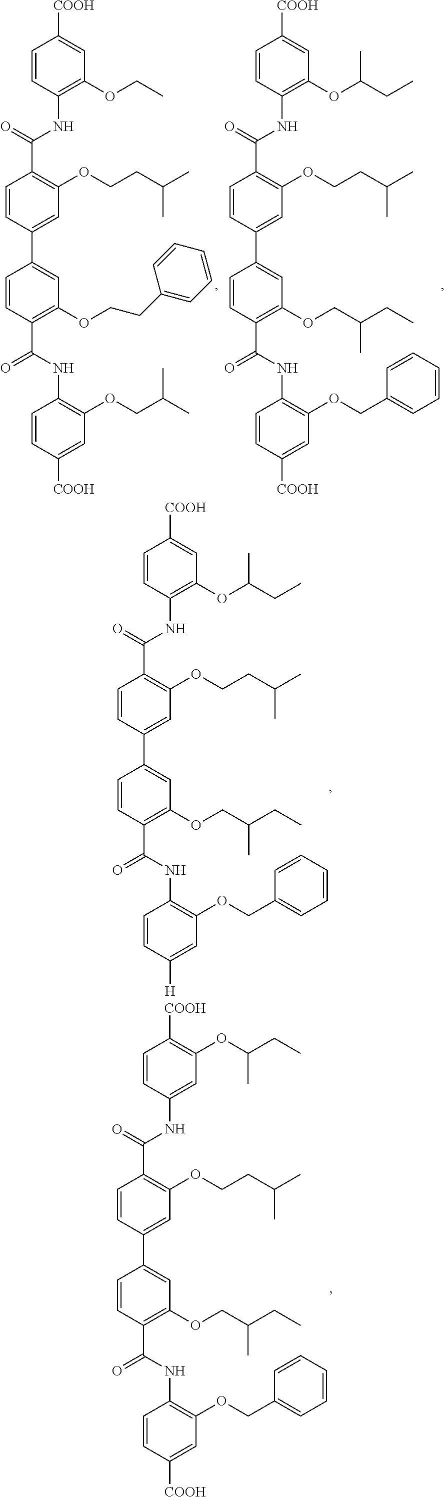

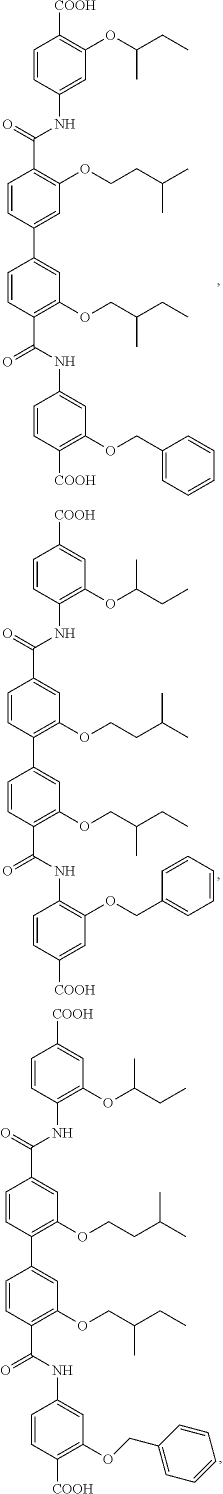

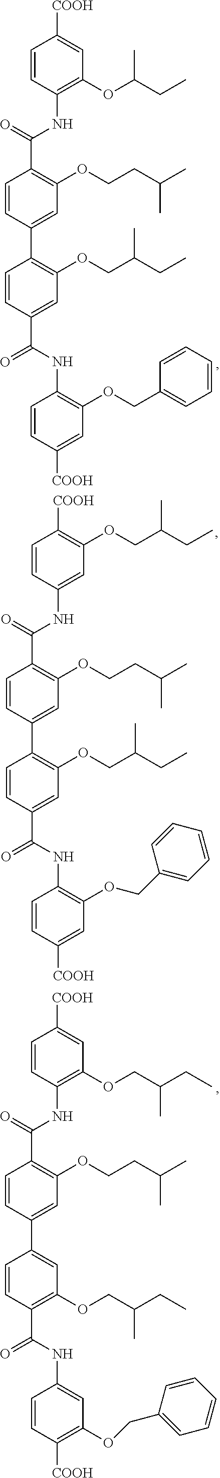

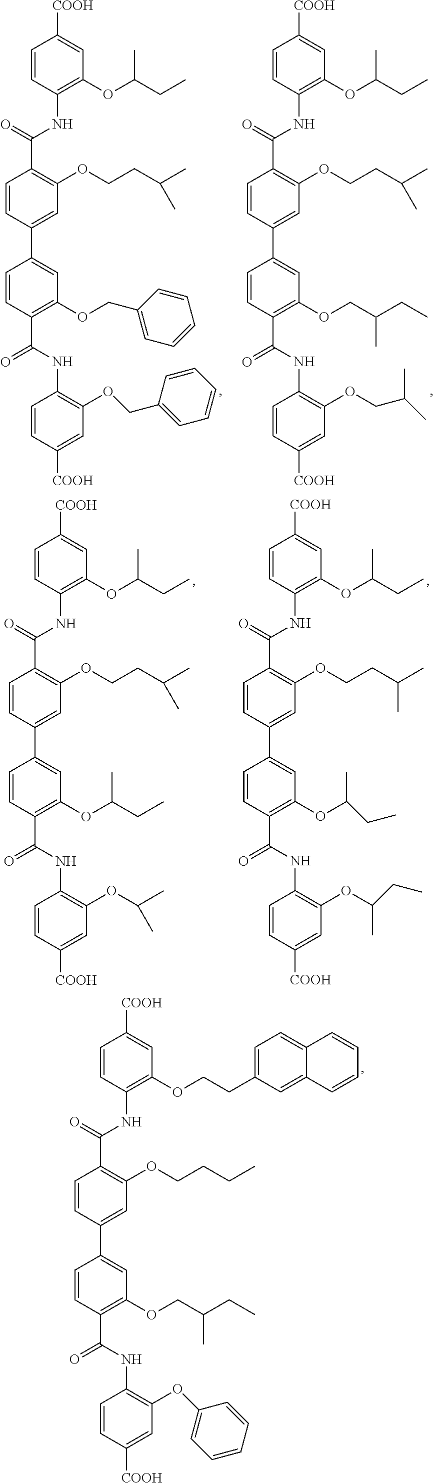

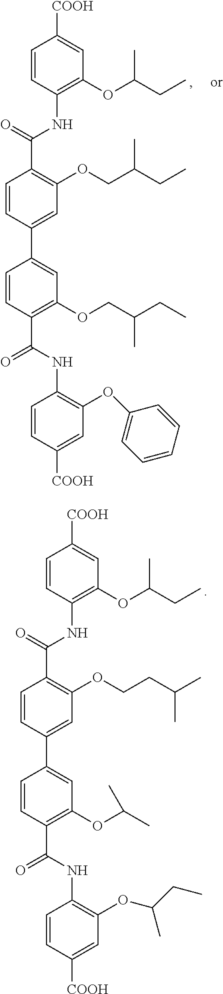

9. The compound of claim 1, wherein the compound binds to at least one anti-apoptotic protein.

10. The compound of claim 9, wherein the at least one anti-apoptotic protein comprises Bcl-xL, Bcl-2, Mcl-1, or any combination thereof.

11. The compound of claim 10, wherein the compound has an IC₅₀ for Bcl-xL of less than about 10 μM.

12. The compound of claim 10, wherein the compound has an IC₅₀ for Bcl-2 of less than about 10 μM.

13. The compound of claim 10, wherein the compound has an IC₅₀ for Mcl-1 of less than about 10 μM.

14. The compound of claim 9, wherein the compound inhibits two or more anti-apoptotic proteins.

15. The compound of claim 9, wherein the compound is selective for a single anti-apoptotic protein.

16. A method for treating cancer in a subject, the method comprising administering a therapeutically effective amount of the compound or pharmaceutically acceptable salt of claim 1 to the subject.

17. The method of claim 16, wherein the cancer comprises prostate cancer, follicular lymphoma, chronic lymphocytic leukemia, acute lymphocytic leukemia, diffuse large B cell lymphoma, multiple myeloma, acute myeloid leukemia, breast cancer, small cell lung cancer, non-small cell lung cancer, ovarian cancer, neuroblastoma, bladder cancer, colorectal cancer, head and neck cancer, or any combination thereof.

18. The method of claim 17, wherein the cancer is prostate cancer.

19. The method of claim 16, wherein the cancer comprises a tumor in the subject and wherein the tumor after treatment has a volume of from at least 5% to at least 50% less than the volume of the tumor before treatment.

20. The method of claim 16, wherein the subject is a mammal.
